(19) United States
(12) Patent Application Publication
(10) Pub. No.: US 2024/0271097 A1
(43) Pub. Date: Aug. 15, 2024

(54) METHODS AND COMPOSITIONS FOR CELL REPROGRAMMING

(71) Applicant: Sanford Burnham Prebys Medical Discovery Institute, La Jolla, CA (US)

(72) Inventors: Alexandre COLAS, La Jolla, CA (US); Maria Azzurra MISSINATO, San Diego, CA (US); Michael S. YU, Cambridge, MA (US)

(21) Appl. No.: 18/568,172

(22) PCT Filed: Jun. 8, 2022
(86) PCT No.: PCT/US2022/032682

§ 371 Date: Dec. 7, 2023

Related U.S. Application Data
(60) Provisional application No. 63/208,414, filed on Jun. 8, 2021.

(51) Int. Cl. C12N 5/077; A61K 35/30; A61K 35/34; C12N 5/0793; C12N 15/113

(52) U.S. Cl. CPC C12N 5/0657; A61K 35/30; A61K 35/34; C12N 5/0619; C12N 15/113; C12N 2310/14; C12N 2501/999; C12N 2506/1307; C12N 2506/28; C12N 2510/00

(57) ABSTRACT

This present disclosure provides methods and pharmaceutical compositions for cell reprogramming and a pharmaceutical composition comprising the reprogrammed cells. In certain embodiments, the method of reprogramming a cell comprises reducing the expression of at least one barrier gene selected from the group consisting of ATF7IP, JUNB, ZNF207, Sp7, FOXA1, HEXIM2, SMARCA5, SOX15, CHST2 or NCEH1, or if the barrier gene is ATF7IP or SOX15, then the expression of a second barrier gene is also reduced. In another embodiment, the reprogramming method comprises reducing the expression of at least two barrier genes selected from the group consisting of ATF7IP, JUNB, ZNF207 and Sp7.

### CROSS-REFERENCE

This patent application claims the benefit of U.S. Provisional Patent Application 63/208,414, filed on Jun. 8, 2021, which is hereby incorporated by reference in its entirety.

### STATEMENT AS TO FEDERALLY SPONSPORED RESEARCH

This invention was made with government support under DISC2-10110 awarded by California Institute for Regenerative Medicine and under R01 HL149992 and R01 HL148827 awarded by National Institutes of Health. The government has certain rights in the invention.

### BACKGROUND

Direct lineage reprogramming of a differentiated and specialized cell type into another, is a major goal of regenerative medicine. The process of reprogramming can be elicited by overexpression of lineage-determining factors (LDFs) (e.g. transcription factors (TFs) and/or miRNAs), which have been used to convert differentiated cells, such as fibroblasts, into induced pluripotent stem cells, neurons, and cardiomyocytes. However, in this context, most studies have reported that only a small fraction of fate-challenged cells undergo reprogramming, thus currently limiting clinical applications.

### SUMMARY

In an aspect, the present disclosure provides a method for generating a cell of interest from a cell of a different cell type. The method comprises reducing expression level(s) of at least one barrier gene selected from the group consisting of ATF7IP, JUNB, ZNF207, Sp7, FOXA1, HEXIM2, SMARCA5, SOX15, CHST2 and NCEH1 in said cell of a different cell type compared to a wild-type cell of said cell of a different cell type; wherein when said at least one barrier gene is ATF7IP or SOX15, at least a second barrier gene is included.

In another aspect, the present disclosure provides a method for generating a cell of interest from a cell of a different cell type. The method comprises reducing expression level(s) of at least two barrier genes selected from the group consisting of ATF7IP, JUNB, ZNF207, and Sp7 in said cell of a different cell type compared to a wild-type cell of said cell of a different cell type.

In some embodiments, the at least one barrier gene comprises ATF7IP, JUNB, and ZNF207. In some embodiments, the at least one barrier gene further comprises Sp7. In some embodiments, the method further comprises increasing an expression level of at least one reprogramming agonist gene in said cell of a different cell type compared to a wild-type cell of said cell of a different cell type.

In some embodiments, the at least one reprogramming agonist gene is selected from the group consisting of MEF2C, HSPB3, TPP1, EMC1, PPIC, IL7R, OLFML3, TCTA, and EFHD1. In some embodiments, the method further comprises increasing an expression level of at least one lineage-determining factor.

In some embodiments, the expression level of said at least one lineage-determining factor is increased within 72 hours after said expression level(s) of said at least one barrier gene is reduced. In some embodiments, the expression level of said at least one lineage-determining factor is increased within 48 hours after said expression level(s) of said at least one barrier gene is reduced. In some embodiments, the expression level of said at least one lineage-determining factor is increased within 24 hours after said expression level(s) of said at least one barrier gene is reduced.

In some embodiments, the cell of a different cell type is a fibroblast, an endothelial cell, or a PBMC.

In some embodiments, the method further comprises altering chromatin structure of said cell of a different cell type compared to a wild-type cell of said cell of a different cell type.

In some embodiments, the cell of a different cell type has a reduced number of motifs bound to a protein expressed by said at least one barrier gene compared to a wild-type cell of said cell of a different cell type.

In some embodiments, the motifs comprise an AP-1 motif.

In some embodiments, the cell of interest is a cardiomyocyte-like cell, a cardiomyocyte, a neuron, a skeletal muscle cell, or an induced pluripotent stem cell.

In some embodiments, the at least one lineage-determining factor is selected from the group consisting of MEF2C, GATA4, TBX5, ASCL1, BRN2, MYTL1, OCT4, KLF4, SOX2, MYC, and MYOD.

In some embodiments, the cell of interest has an increased expression of at least one cardiac marker as compared to said cell of a different cell type. In some embodiments, the at least one cardiac marker is selected from the group consisting of ACTC1, MYL7, TNNT2, SCN5A, RYR2, NPPA, and NPPB. In some embodiments, the cell of interest has an increased expression of at least one stem cell marker as compared to said cell of a different cell type. In some embodiments, the at least one stem cell marker is selected from the group consisting of SSEA4 and NANOG.

In some embodiments, the cell of interest has an increased expression of at least one neuron-specific marker as compared to said cell of a different cell type. In some embodiments, the at least one neuron-specific marker is selected from the group consisting of vGLUT2, GAD67, PVALB, and SYN1.

In some embodiments, the reducing expression level(s) of at least one barrier gene comprises contacting said cell of a different cell type with at least one siRNA molecule targeting said at least one barrier gene.

In some embodiments, the cell of a different cell type is a somatic cell. In some embodiments, the cell of interest is a somatic cell. In some embodiments, the method further comprises converting said cell of a different cell type into an induced pluripotent stem cell (iPSC) state.

In another aspect, the present disclosure provides a pharmaceutical composition, comprising at least one inhibitor of at least one barrier gene selected from the group consisting of ATF7IP, JUNB, ZNF207, Sp7, FOXA1, HEXIM2, SMARCA5, SOX15, CHST2 and NCEH1, and a pharmaceutically acceptable carrier or excipient; wherein when said at least one barrier gene is ATF7IP or SOX15, at least a second inhibitor of a second barrier gene is included.

A pharmaceutical composition, comprising at least one inhibitor of at least two barrier gene selected from the group consisting of ATF7IP, JUNB, ZNF207, and Sp7, and a pharmaceutically acceptable carrier or excipient.

In some embodiments, the at least one barrier gene comprises ATF7IP, JUNB, and ZNF207. In some embodiments, the at least one barrier gene further comprises Sp7. In some embodiments, the at least one inhibitor comprises at least one siRNA molecule targeting said at least one barrier gene.

In another aspect, the present disclosure provides a method for repairing or restoring functional and structural integrity to a damaged tissue in a subject in need thereof, comprising administering to said subject an effective amount of the pharmaceutical composition disclosed herein.

The method of claim 32, further comprising administering at least one lineage-determining factor.

In some embodiments, the one lineage-determining factor is administered within 72 hours after said pharmaceutical composition is administered. In some embodiments, the at least one lineage-determining factor is administered within 48 hours after said pharmaceutical composition is administered. In some embodiments, the at least one lineage-determining factor is administered within 24 hours after said pharmaceutical composition is administered. In some embodiments, the damaged tissue is a cardiac tissue and wherein said at least one lineage-determining factor is MEF2C, GATA4, or TBX5.

In some embodiments, the damaged tissue is a neural tissue and wherein said at least one lineage-determining factor is ASCL1, BRN2, or MYTL1. In some embodiments, the damaged tissue is a skeletal muscle tissue and wherein said at least one lineage-determining factor is MYOD. In some embodiments, the damaged tissue is a stem cell lineage and wherein said at least one lineage-determining factor is OCT4, KLF4, SOX2, or MYC.

In another aspect, the present disclosure provides method for performing cell transplantation in a recipient in need thereof, comprising generating a second cell from a first cell of a donor, wherein said donor is immunocompatible with said recipient, and wherein said second cell is generated according to the method disclosed herein; and transplanting said second cell into said recipient.

In some embodiments, the recipient and said donor are the same individual. In some embodiments, the first cell is a fibroblast or an endothelial cell. In some embodiments, the second cell is a cardiomyocyte-like cell, a cardiomyocyte, a neuron, a skeletal muscle cell, or a stem cell-like cell.

In another aspect, the present disclosure provides methods for generating a cell of interest from a cell of a different cell type, comprising reducing expression level(s) of at least one barrier gene selected from the group consisting of ATF7IP, JUNB, ZNF207, Sp7, FOXA1, HEXIM2, SMARCA5, SOX15, CHST2 and NCEH1 in the cell of a different cell type compared to a wild-type cell of the cell of a different cell type; wherein when the at least one barrier gene is ATF7IP or SOX15, at least a second barrier gene is included.

In another aspect, the present disclosure provides a pharmaceutical compsotion comprising a first plurality of cells having a first cell type and media; made by a process comprising: obtaining a second plurality of cells of a different cell type compared to the first cell type; reducing expression level(s) of at least one barrier gene selected from the group consisting of ATF7IP, JUNB, ZNF207, Sp7, FOXA1, HEXIM2, SMARCA5, SOX15, CHST2 and NCEH1 in the second plurality of cells; wherein when said at least one barrier gene is ATF7IP or SOX15, at least a second barrier gene is included, or reducing expression level(s) of at least two barrier genes selected from the group consisting of ATF7IP, JUNB, ZNF207, and Sp7 in the second plurality of cells.

In some embodiments, the at least one barrier gene comprises ATF7IP, JUNB, and ZNF207. In some embodiments, the at least one barrier gene further comprises Sp7. In some embodiments, the process further comprises increasing an expression level of at least one reprogramming agonist gene in the second plurality of cells. In some embodiments, the at least one reprogramming agonist gene is selected from the group consisting ofMEF2C, HSPB3, TPP1, EMC1, PPIC, IL7R, OLFML3, TCTA, and EFHD1. In some embodiments, the process further comprising increasing an expression level of at least one lineage-determining factor in the first plurality of cells. In some embodiments, the expression level of the at least one lineage-determining factor is increased within 72 hours after the expression level(s) of the at least one barrier gene is reduced. In some embodiments, the expression level of the at least one lineage-determining factor is increased within 48 hours after the expression level(s) of the at least one barrier gene is reduced. In some embodiments, the expression level of the at least one lineage-determining factor is increased within 24 hours after the expression level(s) of the at least one barrier gene is reduced. In some embodiments, the second plurality of cells is a fibroblast, an endothelial cell, or a PBMC. In some embodiments, the process further comprising altering chromatin structure of the second plurality of cells. In some embodiments, the second plurality of cells has a reduced number of motifs bound to a protein expressed by said at least one barrier gene compared to a wild-type cell of the second plurality of cells. In some embodiments, the motifs comprise an AP-1 motif. In some embodiments, the first plurality of cells is a cardiomyocyte-like cell, a cardiomyocyte, a neuron, a skeletal muscle cell, or an induced pluripotent stem cell. In some embodiments, the at least one lineage-determining factor is selected from the group consisting of MEF2C, GATA4, TBX5, ASCL1, BRN2, MYTL1, OCT4, KLF4, SOX2, MYC, and MYOD. In some embodiments, the first plurality of cells has an increased expression of at least one cardiac marker as compared to the second plurality of cells. In some embodiments, the at least one cardiac marker is selected from the group consisting of ACTC1, MYL7, TNNT2, SCN5A, RYR2, NPPA, and NPPB. In some embodiments, the first plurality of cells has an increased expression of at least one stem cell marker as compared to the second plurality of cells. In some embodiments, the at least one stem cell marker is selected from the group consisting of SSEA4 and NANOG. In some embodiments, the first plurality of cells has an increased expression of at least one neuron-specific marker as compared to the second plurality of cells. In some embodiments, the at least one neuron-specific marker is selected from the group consisting of vGLUT2, GAD67, PVALB, and SYN1. In some embodiments, the reducing expression level(s) of at least one barrier gene comprises contacting the second plurality of cells with at least one siRNA molecule targeting said at least one barrier gene. In some embodiments, the second plurality of cells are somatic cells. In some embodiments, the first plurality of cells are somatic cells. In some embodiments, the process further comprising converting the second plurality of cells into an induced pluripotent stem cell (iPSC) state.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawing embodiments, which are presently exemplified. It should be understood, however, that the invention is not limited to the precise arrangement and instrumentalities of the embodiments shown in the drawings.

FIG. 1 illustrates the high-throughput siRNA screening used to identify Atf7ip, JunB, Sp7, and Zfp207 (ZNF207) as barriers (AJSZ) to cell fate reprogramming. FIG. 1a is a schematic of the experimental set-up for the cardiac reprogramming screening assay. iMGT-MEFs carrying the cardiac reporter Myh6-eGFP were transfected with a genome-wide TF siRNA library (1435 TFs) on day −1, treated with Dox on day 0 to induce MGT expression, and collected on day 3 for analysis by confocal fluorescence microscopy. Reprogramming efficiency was quantified as the percentage of Myh6-eGFP+ cells (induced cardiomyocytes, iCMs) on day 3, normalized to the siControl condition. FIG. 1b shows the volcano plot of the library screening results, with the top 20 siRNA hits indicated. N=4. FIG. 1c illustrates the secondary screening; of the top 20 hits with independent siRNAs resulted in the validation and identification of 8 TFs as novel barriers of cardiac reprogramming. Mean±SEM, n=4. FIG. 1d illustrates representative images of siATF7IP- and siControl-transfected iMGT-MEFs on day 3 after MGT induction. Myh6-eGFP is shown in green, cell nuclei are stained blue (DAPI, top right insets). FIG. 1e is a schematic of the experimental set-up for combinatorial screening of the 8 candidate barrier TFs. A total of 255 combinations were tested, each in quadruplicate. FIG. 1f shows the volcano plot of the combinatorial siRNA screening results. Data were normalized to the most effective single barrier TF (siAtf7ip). 1-4 indicate the top combinations compared with siAtf7ip alone (p<0.05). siAtf7ip, siJunb, siSp7, and siZfp207 (siAJSZ) are shown in red. siAJSZ and siControl were the most and least effective combinations, respectively. FIG. 1g is the secondary screening of the top 4 siRNA combinations with independent siRNAs. Single letters are as indicated in FIG. 1f. FIG. 1h illustrates representative images of iMGT-MEFs transfected with siAJSZ (siAtf7ip, siJiub, siSp7, siZfp207) on day 3 after MGT induction. Labeling is as described for (d). FIG. 1i shows quantification of HDFs expressing the cardiac marker ACTN1 30 days after induction of mMGT overexpression. Representative images of HDFs treated as in FIG. 1j and immunostained for the fibroblast marker transgelin (TAGLN, red) and the cardiac marker α-actinin (ACTN1, green). Cell nuclei are stained blue (DAPI). White arrows indicate reprogrammed iCMs. FIG. 1k illustrates quantification of HDFs expressing the neuronal markers MAP2 (green) and TUJ1 (red) 3 days after overexpression of Ascl1, Brn2, and Mytl1 (ABM). FIG. 1l illustrates representative images of HDFs treated as in (k) and inmunostained for MAP2 (green) and TUJ1 (red). Cell nuclei are stained blue (DAPI, top left insets). White arrows indicate TUJ1 MAP2+ double-positive cells. FIG. 1m illustrates quantification ofIHAECs expressing ACTN1 20 days after MGT overexpression. FIG. 1n shows representative images of HAECs treated as in (FIG. 1m) and stained for PECAM1 (red, endothelial marker) and ACTN1 (green). Cells nuclei are stained blue (DAPI, top left inserts). White arrows indicate ACTN1+iCMs. Scale bars: 50 μm. Student's t-test: *p<0.05, **p<0.01, ***p<0.001, ****p<0.0001.

FIG. 2 illustrates that AJSZ regulate chromatin accessibility during cell fate reprogramming. FIG. 2a is a schematic showing a hypothetic role of AJSZ during cardiac reprogramming of HDFs. FIG. 2b is the ChIP-seq results indicating the percentage binding of A, J, S, and Z proteins to the indicated DNA loci. FIG. 2c shows that the most enriched DNA motifs bound by A, J, S, and Z in HDFs. JUNB and ZNF207 share the same binding motif. FIGS. 2d-2f illustrate t-SNE visualization of 7 cell clusters after scATAC-seq of untransfected (ground state) HDFs (FIG. 2d) siControl-transfected HDFs 2 days after mMGT overexpression (FIG. 2e), or siAJSZ-transfected HDFs 2 days after MGT overexpression (FIG. 2f). In contrast to the homogeneity of cells in the ground state (FIG. 2d) or after MGT induction alone (FIG. 2e), knockdown of AJSZ prior to MGT overexpression (FIG. 2f) results in an open chromatin cardiac cluster (#2, black arrow). FIG. 2g indicates the density of domain 1 and 2 sites throughout the genome. Domain 1 represents open chromatin in MGT+siAJSZ cells in cluster #2 that is closed in both ground state fibroblasts and MGT+siControl cells. Conversely, domain 2 is open in both ground state fibroblasts and MGT+siControl cells but closed in MGT+sjAJSZ. FIG. 2h illustrates the distribution of domain 1 and 2 region lengths indicating most regions are 200 to 500 bp long. FIG. 2i illustrates the density of AJSZ binding from the ChIP-seq data in domain 1 and 2. Domain 2 is highly enriched for AJSZ binding sites. FIG. 2j illustrates comparison of motif enrichment between Domains 1 and 2. Motif analysis of domain 1 indicates MEF2 family members bind. Motifs in domain 2 are enriched for factors that bind AP-1 motif. FIG. 2k illustrates a model in which genome-wide binding of AJSZ to AP-1 motifs in domain 2 chromatin impairs the ability of the LDF MEF2C to remodel chromatin and promote domain 1 opening, thereby opposing cell fate conversion.

FIG. 3 illustrates that AJSZ differentially regulate gene expression cell fate reprogramming. FIG. 3a is the heatmap of differentially expressed (DE) genes in siControl- and siAJSZ-transfected HDFs 2 days after MGT overexpression. FIG. 3b is the Venn diagram showing overlap between DE and core promoter bound (−1 kb-TSS-+0.1 kb) genes. 460 genes were both DE and bound by AJSZ at core promoter regions, including 348 downregulated and 112 upregulated genes. FIG. 3c are bar charts showing top ranked biological process terms enriched for the 460 DE and core promoter bound genes. FIG. 3d illustrates the breakdown of the percentage of DE and core promoter bound genes containing A, J, S, and/or Z binding sites. FIG. 3e illustrates ChIP-seq track for JUNB at JUNB binding sites. FIGS. 3f-3g are genome browser views showing binding of JUNB at TAGLN (FIG. 3f) and at MEF2C core promoter regions (FIG. 3g) in HDFs.

FIG. 4 illustrates that AJSZ epigenetically control expression of downstream barriers and agonists of cell fate reprogramming. FIG. 4a is a schematic showing the hypothesis that AJSZ positively regulate expression of reprogramming barriers. FIG. 4b is the volcano plot showing the screening results for siRNAs directed against top 123 downregulated (MGT+siAJSZ vs MGT) and core promoter bound genes in the iMGT-MEF CR assay. The top 3 candidate barrier genes are circled. N=4 per condition. FIG. 4c is a histogram showing validation of siNceh1 and siChst2 effect on CR. FIG. 4d shows representative images for siControl, siChst2 and siNceh1 conditions. Myh6-eGFP+ cells are shown in green and cell nuclei are stained blue (DAPI, top right insets). FIG. 4e is a schematic of the hypothesis that AJSZ negatively regulate reprogramming agonists in HDFs. FIG. 4f is the volcano plot showing the screening results for siRNAs directed against top 77 upregulated (MGT+siAJSZ vs MGT) and core promoter bound genes in siAJSZ-induced iMGT-MEF CR assay. FIG. 4g is a histogram showing validation of top 9 siRNAs that blunt siAJSZ-induced CR without affect cell viability. FIG. 4h shows representative images for siAJSZ+siControl, siMef2c, siHspb3, or siOlfil3 conditions. FIG. 4i is a heatmap summarizing AJSZ expression dependence of identified barriers and agonists in HAECs and HDFs, 2 days after MGT or ABM overexpression. FIG. 4j illustrates a cell fate and cell type-independent model for the AJSZ-mediated transcriptional regulation of cell fate reprogramming. Scale bars: 50 μm. Student's t-test. *p<0.05, **p<0.01, ****p<0.0001.

FIG. 5 illustrates representative images of siControl-transfected with average reprogramming efficiency quantification (6.2% of Myh6-eGFP+ cells) (FIG. 5a) and siControl-, siZfp207-, or siJunb-transfected (FIG. 5b) iMGT-MEFs 3 days after MGT overexpression. The cardiac marker Myh6 is shown in green, and cell nuclei are stained blue (DAPI). Scale bars: 50 μm.

FIG. 6a illustrates immunostaining of untreated HDFs for ATF7IP, JUNB, SP7, or ZNF207 (red) in combination with fibroblast markers TAGLN or vimentin (VIM) (green). Nuclei are stained blue (DAPI). FIG. 6b illustrates qRT-PCR analysis of AJSZ expression in siCTR- or siAJSZ-transfected HDFs 3 days after mMGT overexpression. Data were normalized to the MGT+siCTR cells. FIG. 6c illustrates qRT-PCR analysis of the indicated cardiac gene expression in siCTR- or siAJSZ-transfected HDFs 3 days after mMGT overexpression. FIG. 6d shows immunostaining of ACTN1 and TAGLN in siAJSZ-transfected HDFs analyzed 30 days after MGT overexpression. Lower panels show that some ACTN1+ cells have lost TAGLN staining and show striations. FIG. 6e illustrates fluorescence-based (Fluo-4) quantification of calcium handling in siAJSZ-transfected iMGT-MEF cells 30 days after mMGT overexpression. Scale bars: 50 μm. Student's t-test. *p<0.05, **p<0.01, p***<0.001, ****p<0.0001.

FIG. 7a is a schematic showing the experimental set-up for direct neuronal reprogramming of HDFs with ABM. FIG. 7b is the qRT-PCR analysis of neuronal markers in siRNA-transfected HDFs 3 days after induction of neuronal reprogramming with ABM. FIG. 7c illustrates immunostaining of A, J, S, and Z (red) and the endothelial marker PECAM1 (green) in untreated HAECs. Nuclei were stained with DAPI (blue). Scale bars: 50 μm. FIG. 7e is a schematic showing the experimental set-up for direct cardiac reprogramming of HAECs with mMGT. FIG. 7d shows qRT-PCR analysis of AJSZ expression in siControl- and siAJSZ-transfected HAECs on day XX after mMGT overexpression. FIG. 7f illustrates qPCR of cardiac genes in siControl- and siAJSZ-transfected HAECs on day 3 after mMGT overexpression. Student's t-test. *p<0.05, **p<0.01, ***p<0.001, ****p<0.0001.

FIG. 8a is a schematic of the experimental set-up for determining the optimal timing of barrier gene KD to achieve the most efficient CR of iMGT-MEFs. CR was quantified as the percentage iCMs (Myh6-eGFP+ cells). FIGS. 8b-8c shows quantification (FIG. 8b) and imaging (FIG. 8c) of Myh6-eGFP+ cells (green) in the iMGT-MEF CR assay. Optimal reprogramming was achieved when AJSZ KD was performed 1 day prior to induction of MGT. In contrast, removal of the barriers 1 day after induction of LDFs was ineffective in preventing reprogramming. FIGS. 8d-8e shows quantification (FIG. 8d) and imaging (FIG. 8e) of Myh6-eGFP+ cells (green) in the iMGT-MEF CR assay after AJSZ KD performed 1, 2, or 3 days before induction of MGT, confirming that the optimal timing of KD was day −1. One-way ANOVA, *p<0.05, **p<0.01, ***p<0.001.

FIG. 9a illustrates the summary of the ChIP-seq assay results showing the percentage binding of A, J, S, and Z proteins to the indicated DNA loci. FIG. 9b illustrates the top 3 enriched DNA motifs in A, J, S, and Z binding sites in HDFs. FIG. 9c illustrates all Gene Ontology (GO) terms significantly enriched in cluster 2 HDFs after cardiac reprogramming of MGT+siAJSZ cells. The enriched GO terms are related to cardiac functions. FIG. 9d illustrates ATAC-seq track for NKX2.5 at the TSS regions in HDFs.

FIG. 10a illustrates representative distribution of domain 1 and domain 2 regions at chromosome 4 q28.1. Domain 1 and 2 appear to be intermixed and not gene specific. FIG. 10b illustrates breakdown by percentage of domain 1 and domain 2 site annotation. Both domains show predominantly intronic and intergenic binding. FIG. 10c illustrates the top 3 motifs enriched in domain 2, indicating that all 3 contain the AP-1 motif FIG. 10d illustrates the top motif enriched in domain 1 corresponds to a MEF binding site.

### DETAILED DESCRIPTION

Replacing damaged cells in a patient is the ultimiate goal of regenerative medicine. Transdifferentiation, also called direct cell reprogramming, is one of the potential ways to achieve that goal. The process of transdifferentiation converts an existing differentiated cell into the required cell type. By downregulating one genetic program and upregulating a new genetic program, either simultaneously or subsequently, one cell type may be directly converted into another cell type. Another potential way to achieve the goal is reprogramming. By reprogramming, differentiated cells are induced to reverting back to pluripotentcy. The state of pluripotency allows the cells to be able to differentiate into almost any cell type. (Chris Jopling, Stephanie Boue, et al., Dedifferentiation, transdifferentiation and reprogramming: three rountes to regeneration. Nature Reviews, Molecular Cell Biology, Vol. 12, 79-89 (February 2011)). In other words, transdifferentiation allows for the reprogramming of one somatice cell type directly into another cell lineage, without the need to transition through an induced intermediatry pluripotent state. In typical cellular reprogramming, cells are first converted into an induced pluripotent stem cell (iPSC) state and are then differentiated down a desired lineage to generate a large quantity of reprogrammed cells.

The process of transdifferentiation and/or reprogramming can be elicited by overexpression of lineage-determining factors (LDFs) (e.g. transcription factors (TFs) and/or miRNAs), which have been used to convert differentiated cells, such as fibroblasts, into induced pluripotent stem cells, neurons, and cardiomyocytes. However, in this context, most studies have reported that only a small fraction of fate-challenged cells undergo transdifferentiation and/or reprogramming, thus currently limiting clinical applications. Thus, there is a need for an enhanced transdifferentiation and/or reprogramming. This disclosure relates generally to methods for enhanced cell reprogramming and enhanced cell transdifferentiation. Particularly, disclosed herein are methods for generating a cell of interest from a cell of a different cell type, pharmaceutical compositions, methods for repairing or restoring functional and structural integrity to a damaged tissue in a subject in need thereof, and methods for performing cell transplantation in a recipient in need thereof.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

As used herein, the term “direct reprogramming” or “transdifferentiation” refers to a process where cells convert from one differentiated cell type to another without undergoing an intermediate pluripotent state or progenitor cell type. For example, the cell phenotype of a differentiated cell (e.g., a fibroblast cell) is altered to a different differentiated cell (a cardiomyocyte), without the fibroblast cell being completely reprogrammed to a less differentiated phenotype intermediate (e.g., an induced pluripotent stem cell).

As used herein, the term “cell type” refers to distinct morphological or functional forms of cells.

As used herein, the term “reprogramming” refers to a process that alters or reverses the differentiation status of a somatic cell that is either partially or terminally differentiated. Reprogramming may be partial or complete, that is, the differentiation status of a somatic cell may be partially or completely reversed. In some embodiments, reprogramming is complete when a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

As used herein the term “pluripotent stem cells” (PS cells) are cells that are capable under the right conditions of producing progeny of several different cell types. PS cells are capable of producing progeny that are derivatives of each of the three germ layers: endoderm, mesoderm, and ectoderm, according to a standard art-accepted test, such as the ability to form a teratoma in a suitable host, or the ability to differentiate into cells stainable for markers representing tissue types of all three germ layers in culture. Included in the definition of PS cells are embryonic cells of various types, such as embryonic stem (ES) cells, as well as induced pluripotent stem cells (iPS) that have been reprogrammed from an adult somatic cell.

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally occurring or genetically modified.

As used herein, the term “lineage determining factor” or “LDF” refers to a transcription factor (TF) or a miRNA that regulates lineage decisions and determines cell fates.

As used herein, the term “AJSZ” refers to the four TFs: ATF7IP, JUNB, Sp7, and ZNF207. The term “siAJSZ” refers to a combination of siRNAs targeting AJSZ, with each siRNA targeting one of AJSZ genes.

As used herein, the term “ABM” refers to genes Ascl1, Brn2, and Mytl1.

As used herein, the term “MGT” refers to genes Mef2c, Gata4, and Tbx5.

As used herein, the term “reprogramming agonist gene” or “agonist gene” refers to a gene that encodes a protein that can trigger or induce cell reprogramming. As used herein, the term “reprogramming agonist” or “agonist” refers to a protein that trigger or induce cell reprogramming.

As used herein, the term “reprogramming barrier gene” or “barrier gene” refers to a gene that encodes a protein that can prevent or inhibit cell reprogramming. As used herein, the term “reprogramming barrier” or “barrier” refers to a protein that can prevent or inhibit cell reprogramming.

### Methods of Reprogramming and Transdifferentiation

The present disclosure provides methods for enhanced cell reprogramming and enhanced cell transdifferentiation.

In one aspect, the present disclosure provides a method for generating a cell of interest from a cell of a different cell type, comprising reducing expression level(s) of at least one barrier gene selected from the group consisting of ATF7IP, JUNB, ZNF207, Sp7, FOXA1, HEXIM2, SMARCA5, SOX15, CHST2 and NCEH1 in said cell of a different cell type compared to a wild-type cell of said cell of a different cell type; wherein when said at least one barrier gene is ATF7IP or SOX15, at least a second barrier gene is included. In some embodiments, the at least one barrier gene comprises ATF7IP and a second barrier gene that is selected from the group consisting of JUNB, ZNF207, Sp7, FOXA1, HEXIM2, SMARCA5, SOX15, CHST2 and NCEH1. In some embodiments, the at least one barrier gene comprises JUNB and a second barrier gene that is selected from the group consisting of ATF7IP, ZNF207, Sp7, FOXA1, HEXIM2, SMARCA5, SOX15, CHST2 and NCEH1. In some embodiments, the at least one barrier gene comprises ZNF207 and a second barrier gene that is selected from the group consisting of ATF7IP, JUNB, Sp7, FOXA1, HEXIM2, SMARCA5, SOX15, CHST2 and NCEH1. In some embodiments, the at least one barrier gene comprises Sp7 and a second barrier gene that is selected from the group consisting of ATF7IP, JUNB, ZNF207, FOXA1, HEXIM2, SMARCA5, SOX15, CHST2 and NCEH1. In some embodiments, the at least one barrier gene comprises FOXA1 and a second barrier gene that is selected from the group consisting of ATF7IP, JUNB, ZNF207, Sp7, HEXIM2, SMARCA5, SOX15, CHST2 and NCEH1. In some embodiments, the at least one barrier gene comprises HEXIM2 and a second barrier gene that is selected from the group consisting of ATF7IP, JUNB, ZNF207, Sp7, FOXA1, SMARCA5, SOX15, CHST2 and NCEH1. In some embodiments, the at least one barrier gene comprises SMARCA5 and a second barrier gene that is selected from the group consisting of ATF7IP, JUNB, ZNF207, Sp7, FOXA1, HEXIM2, SOX15, CHST2 and NCEH1. In some embodiments, the at least one barrier gene comprises SOX15 and a second barrier gene that is selected from the group consisting of ATF7IP, JUNB, ZNF207, Sp7, FOXA1, HEXIM2, SMARCA5, CHST2 and NCEH1. In some embodiments, the at least one barrier gene comprises CHST2 and a second barrier gene that is selected from the group consisting of ATF7IP, JUNB, ZNF207, Sp7, FOXA1, HEXIM2, SMARCA5, SOX15 and NCEH1. In some embodiments, the at least one barrier gene comprises NCEH1 and a second barrier gene that is selected from the group consisting of ATF7IP, JUNB, ZNF207, Sp7, FOXA1, HEXIM2, SMARCA5, SOX15 and CHST2. In some embodiments, the at least one barrier gene comprises ATF7IP and JUNB. In some embodiments, the at least one barrier gene comprises ATF7IP and ZNF207. In some embodiments, the at least one barrier gene comprises ATF7IP and Sp7. In some embodiments, the at least one barrier gene comprises ATF7IP and FOXA1. In some embodiments, the at least one barrier gene comprises ATF7IP and HEXIM2. In some embodiments, the at least one barrier gene comprises ATF7IP and SMARCA5. In some embodiments, the at least one barrier gene comprises ATF7IP and SOX15. In some embodiments, the at least one barrier gene comprises ATF7IP and CHST2. In some embodiments, the at least one barrier gene comprises ATF7IP and NCEH1. In some embodiments, the at least one barrier gene comprises JUNB and ZNF207. In some embodiments, the at least one barrier gene comprises JUNB and Sp7. In some embodiments, the at least one barrier gene comprises JUNB and FOXA1. In some embodiments, the at least one barrier gene comprises JUNB and HEXIM2. In some embodiments, the at least one barrier gene comprises JUNB and SMARCA5. In some embodiments, the at least one barrier gene comprises JUNB and SOX15. In some embodiments, the at least one barrier gene comprises JUNB and CHST2. In some embodiments, the at least one barrier gene comprises JUNB and NCEH1. In some embodiments, the at least one barrier gene comprises ZNF207 and Sp7. In some embodiments, the at least one barrier gene comprises ZNF207 and FOXA1. In some embodiments, the at least one barrier gene comprises ZNF207 and HEXIM2. In some embodiments, the at least one barrier gene comprises ZNF207 and SMARCA5. In some embodiments, the at least one barrier gene comprises ZNF207 and SOX15. In some embodiments, the at least one barrier gene comprises ZNF207 and CHST2. In some embodiments, the at least one barrier gene comprises ZNF207 and NCEH1. In some embodiments, the at least one barrier gene comprises Sp7 and FOXA1. In some embodiments, the at least one barrier gene comprises Sp7 and HEXIM2. In some embodiments, the at least one barrier gene comprises Sp7 and SMARCA5. In some embodiments, the at least one barrier gene comprises Sp7 and SOX15. In some embodiments, the at least one barrier gene comprises Sp7 and CHST2. In some embodiments, the at least one barrier gene comprises Sp7 and NCEH1. In some embodiments, the at least one barrier gene comprises FOXA1 and HEXIM. In some embodiments, the at least one barrier gene comprises FOXA1 and SMARCA5. In some embodiments, the at least one barrier gene comprises FOXA1 and SOX15. In some embodiments, the at least one barrier gene comprises FOXA1 and CHST2. In some embodiments, the at least one barrier gene comprises FOXA1 and NCEH1. In some embodiments, the at least one barrier gene comprises HEXIM2 and SMARCA5. In some embodiments, the at least one barrier gene comprises HEXIM2 and SOX15. In some embodiments, the at least one barrier gene comprises HEXIM2 and CHST2. In some embodiments, the at least one barrier gene comprises HEXIM2 and NCEH1. In some embodiments, the at least one barrier gene comprises SMARCA5 and SOX15. In some embodiments, the at least one barrier gene comprises SMARCA5 and CHST2. In some embodiments, the at least one barrier gene comprises SMARCA5 and NCEH1. In some embodiments, the at least one barrier gene comprises SOX15 and CHST2. In some embodiments, the at least one barrier gene comprises SOX15 and NCEH1. In some embodiments, the at least one barrier gene comprises CHST2 and NCEH1.

In some embodiments, the at least one barrier gene comprises ATF7IP and JUNB. In some embodiments, the at least one barrier gene comprises ATF7IP and ZNF207. In some embodiments, the at least one barrier gene comprises ATF7IP and Sp7. In some embodiments, the at least one barrier gene comprises ATF7IP and FOXA1. In some embodiments, the at least one barrier gene comprises ATF7IP and HEXIM2. In some embodiments, the at least one barrier gene comprises ATF7IP and SMARCA5. In some embodiments, the at least one barrier gene comprises ATF7IP and SOX15. In some embodiments, the at least one barrier gene comprises ATF7IP and CHST2. In some embodiments, the at least one barrier gene comprises ATF7IP and NCEH1. In some embodiments, the at least one barrier gene comprises JUNB and ZNF207. In some embodiments, the at least one barrier gene comprises JUNB and Sp7. In some embodiments, the at least one barrier gene comprises JUNB and FOXA1. In some embodiments, the at least one barrier gene comprises JUNB and HEXIM2. In some embodiments, the at least one barrier gene comprises JUNB and SMARCA5. In some embodiments, the at least one barrier gene comprises JUNB and SOX15. In some embodiments, the at least one barrier gene comprises JUNB and CHST2. In some embodiments, the at least one barrier gene comprises JUNB and NCEH1. In some embodiments, the at least one barrier gene comprises ZNF207 and Sp7. In some embodiments, the at least one barrier gene comprises ZNF207 and FOXA1. In some embodiments, the at least one barrier gene comprises ZNF207 and HEXIM2. In some embodiments, the at least one barrier gene comprises ZNF207 and SMARCA5. In some embodiments, the at least one barrier gene comprises ZNF207 and SOX15. In some embodiments, the at least one barrier gene comprises ZNF207 and CHST2. In some embodiments, the at least one barrier gene comprises ZNF207 and NCEH1. In some embodiments, the at least one barrier gene comprises Sp7 and FOXA1. In some embodiments, the at least one barrier gene comprises Sp7 and HEXIM2. In some embodiments, the at least one barrier gene comprises Sp7 and SMARCA5. In some embodiments, the at least one barrier gene comprises Sp7 and SOX15. In some embodiments, the at least one barrier gene comprises Sp7 and CHST2. In some embodiments, the at least one barrier gene comprises Sp7 and NCEH1. In some embodiments, the at least one barrier gene comprises FOXA1 and HEXIM2. In some embodiments, the at least one barrier gene comprises FOXA1 and SMARCA5. In some embodiments, the at least one barrier gene comprises FOXA1 and SOX15. In some embodiments, the at least one barrier gene comprises FOXA1 and CHST2. In some embodiments, the at least one barrier gene comprises FOXA1 and NCEH1. In some embodiments, the at least one barrier gene comprises HEXIM2 and SMARCA5. In some embodiments, the at least one barrier gene comprises HEXIM2 and SOX15. In some embodiments, the at least one barrier gene comprises HEXIM2 and CHST2. In some embodiments, the at least one barrier gene comprises HEXIM2 and NCEH1. In some embodiments, the at least one barrier gene comprises SMARCA5 and SOX15. In some embodiments, the at least one barrier gene comprises SMARCA5 and CHST2. In some embodiments, the at least one barrier gene comprises SMARCA5 and NCEH1. In some embodiments, the at least one barrier gene comprises SOX15 and CHST2. In some embodiments, the at least one barrier gene comprises SOX15 and NCEH1. In some embodiments, the at least one barrier gene comprises CHST2 and NCEH1.

In some embodiments, the at least one barrier gene comprises ATF7IP, JUNB, and ZNF207. In some embodiments, the at least one barrier gene comprises ATF7IP, JUNB, and Sp7. In some embodiments, the at least one barrier gene comprises ATF7IP, JUNB, and FOXA1. In some embodiments, the at least one barrier gene comprises ATF7IP, JUNB, and HEXIM2. In some embodiments, the at least one barrier gene comprise ATF7IP, JUNB, and SMARCA5. In some embodiments, the at least one barrier gene comprises ATF7IP, JUNB, and SOX15. In some embodiments, the at least one barrier gene comprise ATF7IP, JUNB, and CHST2. In some embodiments, the at least one barrier gene comprises ATF7IP, JUNB, and NCEH1. In some embodiments, the at least one barrier gene comprises ATF7IP, ZNF207, and Sp7. In some embodiments, the at least one barrier gene comprises ATF7IP, ZNF207, and FOXA1. In some embodiments, the at least one barrier gene comprises ATF7IP, ZNF207, and HEXIM2. In some embodiments, the at least one barrier gene comprises ATF7IP, ZNF207, and SMARCA5. In some embodiments, the at least one barrier gene comprises ATF7IP, ZNF207, and SOX15. In some embodiments, the at least one barrier gene comprises ATF7IP, ZNF207, and CHST2. In some embodiments, the at least one barrier gene comprises ATF7IP, ZNF207, and NCEH1. In some embodiments, the at least one barrier gene comprises ATF7IP, Sp7, and FOXA1. In some embodiments, the at least one barrier gene comprises ATF7IP, Sp7, and HEXIM2. In some embodiments, the at least one barrier gene comprise ATF7IP, Sp7, and SMARCA5. In some embodiments, the at least one barrier gene comprises ATF7IP, Sp7, and SOX15. In some embodiments, the at least one barrier gene comprises ATF7IP, Sp7, and CHST2. In some embodiments, the at least one barrier gene comprises ATF7IP, Sp7, and NCEH1. In some embodiments, the at least one barrier gene comprises ATF7IP, FOXA1, and HEXIM2. In some embodiments, the at least one barrier gene comprises ATF7IP, FOXA1, and SMARCA5. In some embodiments, the at least one barrier gene comprises ATF7IP, FOXA1, and SOX15. In some embodiments, the at least one barrier gene comprises ATF7IP, FOXA1, and CHST2. In some embodiments, the at least one barrier gene comprises ATF7IP, FOXA1, and NCEH1. In some embodiments, the at least one barrier gene comprises ATF7IP, HEXIM2, and SMARCA5. In some embodiments, the at least one barrier gene comprises ATF7IP, HEXIM2, and SOX15. In some embodiments, the at least one barrier gene comprises ATF7IP, HEXIM2, and CHST2. In some embodiments, the at least one barrier gene comprises ATF7IP, HEXIM2, and NCEH1. In some embodiments, the at least one barrier gene comprises ATF7IP, SMARCA5, and SOX15. In some embodiments, the at least one barrier gene comprises ATF7IP, SMARCA5, and CHST2. In some embodiments, the at least one barrier gene comprises ATF7IP, SMARCA5, and NCEH1. In some embodiments, the at least one barrier gene comprises ATF7IP, SOX15, and CHST2. In some embodiments, the at least one barrier gene comprises ATF7IP, SOX15, and NCEH1. In some embodiments, the at least one barrier gene comprises JUNB, ZNF207, and Sp7. In some embodiments, the at least one barrier gene comprises JUNB, ZNF207, and FOXA1. In some embodiments, the at least one barrier gene comprises JUNB, ZNF207, and HEXIM2. In some embodiments, the at least one barrier gene comprises JUNB, ZNF207, and SMARCA5. In some embodiments, the at least one barrier gene comprises JUNB, ZNF207, and SOX15. In some embodiments, the at least one barrier gene comprises JUNB, ZNF207, and CHST2. In some embodiments, the at least one barrier gene comprises JUNB, ZNF207, and NCEH1. In some embodiments, the at least one barrier gene comprises JUNB, Sp7, and FOXA1. In some embodiments, the at least one barrier gene comprises JUNB, Sp7, and HEXIM2. In some embodiments, the at least one barrier gene comprises JUNB, Sp7, and SMARCA5. In some embodiments, the at least one barrier gene comprises JUNB, Sp7, and SOX15. In some embodiments, the at least one barrier gene comprises JUNB, Sp7, and CHST2. In some embodiments, the at least one barrier gene comprises JUNB, Sp7, and NCEH1. In some embodiments, the at least one barrier gene comprises JUNB, FOXA1, and HEXIM2. In some embodiments, the at least one barrier gene comprises JUNB, FOXA1, and SMARCA5. In some embodiments, the at least one barrier gene comprises JUNB, FOXA1, and SOX15. In some embodiments, the at least one barrier gene comprises JUNB, FOXA1, and CHST2. In some embodiments, the at least one barrier gene comprises JUNB, FOXA1, and NCEH1. In some embodiments, the at least one barrier gene comprises JUNB, HEXIM2, and SMARCA5. In some embodiments, the at least one barrier gene comprises JUNB, HEXIM2, and SOX15. In some embodiments, the at least one barrier gene comprises JUNB, HEXIM2, and CHST2. In some embodiments, the at least one barrier gene comprises JUNB, HEXIM2, and NCEH1. In some embodiments, the at least one barrier gene comprises JUNB, SMARCA5, and SOX15. In some embodiments, the at least one barrier gene comprises JUNB, SMARCA5, and CHST2. In some embodiments, the at least one barrier gene comprises JUNB, SMARCA5, and NCEH1. In some embodiments, the at least one barrier gene comprises JUNB, SOX15, and CHST2. In some embodiments, the at least one barrier gene comprises JUNB, SOX15, and NCEH1. In some embodiments, the at least one barrier gene comprises JUNB, CHST2 and NCEH1. In some embodiments, the at least one barrier gene comprises ZNF207, Sp7, and FOXA1. In some embodiments, the at least one barrier gene comprises ZNF207, Sp7, and HEXIM2. In some embodiments, the at least one barrier gene comprises ZNF207, Sp7, and SMARCA5. In some embodiments, the at least one barrier gene comprises ZNF207, Sp7, and SOX15. In some embodiments, the at least one barrier gene comprises ZNF207, Sp7, and CHST2. In some embodiments, the at least one barrier gene comprises ZNF207, Sp7, and NCEH1. In some embodiments, the at least one barrier gene comprises ZNF207, FOXA1, and HEXIM2. In some embodiments, the at least one barrier gene comprises ZNF207, FOXA1, and SMARCA5. In some embodiments, the at least one barrier gene comprises ZNF207, FOXA1, and SOX15. In some embodiments, the at least one barrier gene comprises ZNF207, FOXA1, and CHST2. In some embodiments, the at least one barrier gene comprises ZNF207, FOXA1, and NCEH1. In some embodiments, the at least one barrier gene comprises ZNF207, HEXIM2, and SMARCA5. In some embodiments, the at least one barrier gene comprises ZNF207, HEXIM2, and SOX15. In some embodiments, the at least one barrier gene comprises ZNF207, HEXIM2, and CHST2. In some embodiments, the at least one barrier gene comprises ZNF207, HEXIM2, and NCEH1. In some embodiments, the at least one barrier gene comprises ZNF207, SMARCA5, and SOX15. In some embodiments, the at least one barrier gene comprises ZNF207, SMARCA5, and CHST2. In some embodiments, the at least one barrier gene comprises ZNF207, SMARCA5, and NCEH1. In some embodiments, the at least one barrier gene comprises ZNF207, SOX15, and CHST2. In some embodiments, the at least one barrier gene comprises ZNF207, SOX15, and NCEH1. In some embodiments, the at least one barrier gene comprises ZNF207, CHST2 and NCEH1. In some embodiments, the at least one barrier gene comprises ZNF207, HEXIM2, and SMARCA5. In some embodiments, the at least one barrier gene comprises ZNF207, HEXIM2, and SOX15. In some embodiments, the at least one barrier gene comprises ZNF207, HEXIM2, and CHST2. In some embodiments, the at least one barrier gene comprises ZNF207, HEXIM2, and NCEH1. In some embodiments, the at least one barrier gene comprises ZNF207, SMARCA5, and SOX15. In some embodiments, the at least one barrier gene comprises ZNF207, SMARCA5, and CHST2. In some embodiments, the at least one barrier gene comprises ZNF207, SMARCA5, and NCEH1. In some embodiments, the at least one barrier gene comprises ZNF207, SOX15, and CHST2. In some embodiments, the at least one barrier gene comprises ZNF207, SOX15, and NCEH1. In some embodiments, the at least one barrier gene comprises ZNF207, CHST2 and NCEH1. In some embodiments, the at least one barrier gene comprises Sp7, FOXA1, and HEXIM2. In some embodiments, the at least one barrier gene comprises Sp7, FOXA1, and SMARCA5. In some embodiments, the at least one barrier gene comprises Sp7, FOXA1, and SOX15. In some embodiments, the at least one barrier gene comprises Sp7, FOXA1, and CHST2. In some embodiments, the at least one barrier gene comprises Sp7, FOXA1, and NCEH1. In some embodiments, the at least one barrier gene comprises FOXA1, HEXIM2, and SMARCA5. In some embodiments, the at least one barrier gene comprises FOXA1, HEXIM2, and SOX15. In some embodiments, the at least one barrier gene comprises FOXA1, HEXIM2, and CHST2. In some embodiments, the at least one barrier gene comprises FOXA1, HEXIM2, and NCEH1. In some embodiments, the at least one barrier gene comprises HEXIM2, SMARCA5, and SOX15. In some embodiments, the at least one barrier gene comprises HEXIM2, SMARCA5, and CHST2. In some embodiments, the at least one barrier gene comprises HEXIM2, SMARCA5, and NCEH1. In some embodiments, the at least one barrier gene comprises SMARCA5, SOX15, and CHST2. In some embodiments, the at least one barrier gene comprises SMARCA5, SOX15, and NCEH1. In some embodiments, the at least one barrier gene comprises SOX15, CHST2, and NCEH1.

In another aspect, the present disclosure provides methods for generating a cell of interest from a cell of a different cell type, comprising reducing expression level(s) of at least two barrier genes selected from the group consisting of ATF7IP, JUNB, ZNF207, and Sp7 in said cell of a different cell type compared to a wild-type cell of said cell of a different cell type. In some embodiments, the at least two barrier genes comprise ATF7IP and JUNB. In some embodiments, the at least two barrier genes comprise ATF7IP and ZNF207. In some embodiments, the at least two barrier genes comprise ATF7IP and Sp7. In some embodiments, the at least two barrier genes comprise JUNB and ZNF207. In some embodiments, the at least two barrier genes comprise JUNB and Sp7. In some embodiments, the at least two barrier genes comprise ZNF207 and Sp7.

In some embodiments, the at least two barrier gene comprise ATF7IP, JUNB, and a third barrier gene that is selected from the group consisting of ZNF207, Sp7, FOXA1, HEXIM2, SMARCA5, SOX15, CHST2 and NCEH1. In some embodiments, the at least two barrier gene comprise ATF7IP, JUNB, and ZNF207. In some embodiments, the at least two barrier gene comprise ATF7IP, JUNB, and Sp7. In some embodiments, the at least two barrier gene comprise ATF7IP, JUNB, and FOXA1. In some embodiments, the at least two barrier gene comprise ATF7IP, JUNB, and HEXIM2. In some embodiments, the at least two barrier gene comprise ATF7IP, JUNB, and SMARCA5. In some embodiments, the at least two barrier gene comprise ATF7IP, JUNB, and SOX15. In some embodiments, the at least two barrier gene comprise ATF7IP, JUNB, and CHST2. In some embodiments, the at least two barrier gene comprise ATF7IP, JUNB, and NCEH1.

In some embodiments, the at least two barrier genes comprise ATF7IP, ZNF207, and a third barrier gene that is selected from the group consisting of JUNB, Sp7, FOXA1, HEXIM2, SMARCA5, SOX15, CHST2 and NCEH1. In some embodiments, the at least two barrier genes comprise ATF7IP, ZNF207, and JUNB. In some embodiments, the at least two barrier genes comprise ATF7IP, ZNF207, and Sp7. In some embodiments, the at least two barrier genes comprise ATF7IP, ZNF207, and FOXA1.

In some embodiments, the at least two barrier genes comprise ATF7IP, ZNF207, and HEXIM2. In some embodiments, the at least two barrier genes comprise ATF7IP, ZNF207, and SMARCA5. In some embodiments, the at least two barrier genes comprise ATF7IP, ZNF207, and SOX15. In some embodiments, the at least two barrier genes comprise ATF7IP, ZNF207, and CHST2. In some embodiments, the at least two barrier genes comprise ATF7IP, ZNF207, and NCEH1.

In some embodiments, the at least two barrier genes comprise ATF7IP, Sp7, and a third barrier gene that is selected from the group consisting of JUNB, ZNF207, FOXA1, HEXIM2, SMARCA5, SOX15, CHST2 and NCEH1. In some embodiments, the at least two barrier genes comprise ATF7IP, Sp7, and JUNB. In some embodiments, the at least two barrier genes comprise ATF7IP, Sp7, and ZNF207. In some embodiments, the at least two barrier genes comprise ATF7IP, Sp7, and FOXA1. In some embodiments, the at least two barrier genes comprise ATF7IP, Sp7, and HEXIM2. In some embodiments, the at least two barrier genes comprise ATF7IP, Sp7, and SMARCA5. In some embodiments, the at least two barrier genes comprise ATF7IP, Sp7, and SOX15. In some embodiments, the at least two barrier genes comprise ATF7IP, Sp7, and CHST2. In some embodiments, the at least two barrier genes comprise ATF7IP, Sp7, and NCEH1.

In some embodiments, the at least two barrier genes comprise JUNB, ZNF207, and a third barrier gene that is selected from the group consisting of ATF7IP, Sp7, FOXA1, HEXIM2, SMARCA5, SOX15, CHST2 and NCEH1. In some embodiments, the at least two barrier genes comprise JUNB, ZNF207, and ATF7IP. In some embodiments, the at least two barrier genes comprise JUNB, ZNF207, and Sp7. In some embodiments, the at least two barrier genes comprise JUNB, ZNF207, and FOXA1. In some embodiments, the at least two barrier genes comprise JUNB, ZNF207, and HEXIM2. In some embodiments, the at least two barrier genes comprise JUNB, ZNF207, and SMARCA5. In some embodiments, the at least two barrier genes comprise JUNB, ZNF207, and SOX15. In some embodiments, the at least two barrier genes comprise JUNB, ZNF207, and CHST2. In some embodiments, the at least two barrier genes comprise JUNB, ZNF207, and NCEH1.

In some embodiments, the at least two barrier genes comprise JUNB, Sp7, and a third barrier gene that is selected from the group consisting of ATF7IP, ZNF207, FOXA1, HEXIM2, SMARCA5, SOX15, CHST2 and NCEH1. In some embodiments, the at least two barrier genes comprise JUNB, Sp7, and ATF7IP. In some embodiments, the at least two barrier genes comprise JUNB, Sp7, and ZNF207. In some embodiments, the at least two barrier genes comprise JUNB, Sp7, and FOXA1. In some embodiments, the at least two barrier genes comprise JUNB, Sp7, and HEXIM2. In some embodiments, the at least two barrier genes comprise JUNB, Sp7, and SMARCA5. In some embodiments, the at least two barrier genes comprise JUNB, Sp7, and SOX15. In some embodiments, the at least two barrier genes comprise JUNB, Sp7, and CHST2. In some embodiments, the at least two barrier genes comprise JUNB, Sp7, and NCEH1.

In some embodiments, the at least two barrier genes comprise ZNF207, Sp7, and a third barrier gene that is selected from the group consisting of ATF7IP, JUNB, FOXA1, HEXIM2, SMARCA5, SOX15, CHST2 and NCEH1. In some embodiments, the at least two barrier genes comprise ZNF207, Sp7, and ATF7IP. In some embodiments, the at least two barrier genes comprise ZNF207, Sp7, and JUNB. In some embodiments, the at least two barrier genes comprise ZNF207, Sp7, and FOXA1. In some embodiments, the at least two barrier genes comprise ZNF207, Sp7, and HEXIM2. In some embodiments, the at least two barrier genes comprise ZNF207, Sp7, and SMARCA5. In some embodiments, the at least two barrier genes comprise ZNF207, Sp7, and SOX15. In some embodiments, the at least two barrier genes comprise ZNF207, Sp7, and CHST2. In some embodiments, the at least two barrier genes comprise ZNF207, Sp7, and NCEH1.

In some embodiments, the at least two barrier genes comprise ATF7IP and JUNB. In some embodiments, the at least two barrier genes comprise ATF7IP and ZNF207. In some embodiments, the at least two barrier genes comprise ATF7IP and Sp7. In some embodiments, the at least two barrier genes comprise JUNB and ZNF207. In some embodiments, the at least two barrier genes comprise JUNB and Sp7. In some embodiments, the at least two barrier genes comprise ZNF207 and Sp7. In some embodiments, the at least two barrier genes comprise ATF7IP, JUNB, and ZNF207. In some embodiments, the at least two barrier genes comprise ATF7IP, JUNB, and Sp7. In some embodiments, the at least two barrier genes comprise JUNB, ZNF207, and Sp7. In some embodiments, the at least two barrier genes comprise ATF7IP, ZNF207, and Sp7.

In some embodiments, the at least two barrier genes comprise ATF7IP, JUNB, and ZNF207 and a fourth barrier gene that is selected from the group consisting of Sp7, FOXA1, HEXIM2, SMARCA5, SOX15, CHST2 and NCEH1. In some embodiments, the at least two barrier genes comprise ATF7IP, JUNB, ZNF207 and Sp7. In some embodiments, the at least two barrier genes comprise ATF7IP, JUNB, ZNF207 and FOXA1. In some embodiments, the at least two barrier genes comprise ATF7IP, JUNB, ZNF207 and HEXIM2. In some embodiments, the at least two barrier genes comprise ATF7IP, JUNB, ZNF207 and SMARCA5. In some embodiments, the at least two barrier genes comprise ATF7IP, JUNB, ZNF207 and SOX15. In some embodiments, the at least two barrier genes comprise ATF7IP, JUNB, ZNF207 and CHST2. In some embodiments, the at least two barrier genes comprise ATF7IP, JUNB, ZNF207 and NCEH1.

In some embodiments, the at least two barrier genes comprise ATF7IP, JUNB, and Sp7, and a fourth barrier gene selected from the group consisting of ZNF207, FOXA1, HEXIM2, SMARCA5, SOX15, CHST2 and NCEH1. In some embodiments, the at least two barrier genes comprise ATF7IP, JUNB, Sp7, and ZNF207. In some embodiments, the at least two barrier genes comprise ATF7IP, JUNB, Sp7, and FOXA1. In some embodiments, the at least two barrier genes comprise ATF7IP, JUNB, Sp7, and HEXIM2. In some embodiments, the at least two barrier genes comprise ATF7IP, JUNB, Sp7, and SMARCA5. In some embodiments, the at least two barrier genes comprise ATF7IP, JUNB, Sp7, and SOX15. In some embodiments, the at least two barrier genes comprise ATF7IP, JUNB, Sp7, and CHST2. In some embodiments, the at least two barrier genes comprise ATF7IP, JUNB, Sp7, and NCEH1.

In some embodiments, the at least two barrier genes comprise JUNB, ZNF207, and Sp7, and a fourth barrier gene that is selected from the group consisting of ATF7IP, FOXA1, HEXIM2, SMARCA5, SOX15, CHST2 and NCEH1. In some embodiments, the at least two barrier genes comprise JUNB, ZNF207, Sp7, and ATF7IP. In some embodiments, the at least two barrier genes comprise JUNB, ZNF207, Sp7, and FOXA1. In some embodiments, the at least two barrier genes comprise JUNB, ZNF207, Sp7, and HEXIM2. In some embodiments, the at least two barrier genes comprise JUNB, ZNF207, Sp7, and SMARCA5. In some embodiments, the at least two barrier genes comprise JUNB, ZNF207, Sp7, and SOX15. In some embodiments, the at least two barrier genes comprise JUNB, ZNF207, Sp7, and CHST2. In some embodiments, the at least two barrier genes comprise JUNB, ZNF207, Sp7, and NCEH1.

In some embodiments, the at least two barrier genes comprise ATF7IP, ZNF207, and Sp7, and a fourth barrier gene that is selected from the group consisting of JUNB, FOXA1, HEXIM2, SMARCA5, SOX15, CHST2 and NCEH1. In some embodiments, the at least two barrier genes comprise ATF7IP, ZNF207, Sp7, and JUNB. In some embodiments, the at least two barrier genes comprise ATF7IP, ZNF207, Sp7, and FOXA1. In some embodiments, the at least two barrier genes comprise ATF7IP, ZNF207, Sp7, and HEXIM2. In some embodiments, the at least two barrier genes comprise ATF7IP, ZNF207, Sp7, and SMARCA5. In some embodiments, the at least two barrier genes comprise ATF7IP, ZNF207, Sp7, and SOX15. In some embodiments, the at least two barrier genes comprise ATF7IP, ZNF207, Sp7, and CHST2. In some embodiments, the at least two barrier genes comprise ATF7IP, ZNF207, Sp7, and NCEH1.

In some embodiments, the at least two barrier genes comprise ATF7IP, JUNB, ZNF207 and Sp7. In some embodiments, the at least two barrier genes comprise ATF7IP, JUNB, ZNF207 and Sp7 and a fifth barrier gene that is selected from the group consisting of FOXA1, HEXIM2, SMARCA5, SOX15, CHST2 and NCEH1. In some embodiments, the at least two barrier genes comprise ATF7IP, JUNB, ZNF207, Sp7, and FOXA1. In some embodiments, the at least two barrier genes comprise ATF7IP, JUNB, ZNF207, Sp7, and HEXIM2. In some embodiments, the at least two barrier genes comprise ATF7IP, JUNB, ZNF207, Sp7, and SMARCA5. In some embodiments, the at least two barrier genes comprise ATF7IP, JUNB, ZNF207, Sp7, and SOX15. In some embodiments, the at least two barrier genes comprise ATF7IP, JUNB, ZNF207, Sp7, and CHST2. In some embodiments, the at least two barrier genes comprise ATF7IP, JUNB, ZNF207, Sp7, and NCEH1.

In some embodiments, the at least two barrier genes consist of ATF7IP, JUNB, and ZNF207. In some embodiments, the at least two barrier genes consist of ATF7IP, JUNB, ZNF207 and Sp7.

In some embodiments, the method further comprises increasing an expression level of at least one reprogramming agonist gene in the cell of a different cell type compared to a wild-type cell of the cell of a different cell type. In some embodiments, the at least one reprogramming agonist gene is selected from the group consisting of MEF2C, HSPB3, TPP1, EMC1, PPIC, IL7R, OLFML3, TCTA, and EFHD1. In some embodiments, the at least one reprogramming agonist gene is MEF2C. In some embodiments, the at least one reprogramming agonist gene is HSPB3. In some embodiments, the at least one reprogramming agonist gene is TPP1. In some embodiments, the at least one reprogramming agonist gene is EMC1. In some embodiments, the at least one reprogramming agonist gene is PPIC. In some embodiments, the at least one reprogramming agonist gene is IL7R. In some embodiments, the at least one reprogramming agonist gene is OLFML3. In some embodiments, the at least one reprogramming agonist gene is TCTA. In some embodiments, the at least one reprogramming agonist gene is EFHD1.

In some embodiments, the method further comprises increasing an expression level of at least one lineage-determining factor. In some embodiments, the expression level of the at least one lineage-determining factor is increased within 10 days after reducing the expression level(s) of the at least one barrier gene. In some embodiments, the expression level of the at least one lineage-determining factor is increased within 9 days after reducing the expression level(s) of the at least one barrier gene. In some embodiments, the expression level of the at least one lineage-determining factor is increased within 8 days after reducing the expression level(s) of the at least one barrier gene. In some embodiments, the expression level of the at least one lineage-determining factor is increased within 7 days after reducing the expression level(s) of the at least one barrier gene. In some embodiments, the expression level of the at least one lineage-determining factor is increased within 6 days after reducing the expression level(s) of the at least one barrier gene. In some embodiments, the expression level of the at least one lineage-determining factor is increased within 5 days after reducing the expression level(s) of the at least one barrier gene. In some embodiments, the expression level of the at least one lineage-determining factor is increased within 4 days after reducing the expression level(s) of the at least one barrier gene. In some embodiments, the expression level of the at least one lineage-determining factor is increased within 72 hours after reducing the expression level(s) of the at least one barrier gene. In some embodiments, the expression level of the at least one lineage-determining factor is increased within 60 hours after reducing the expression level(s) of the at least one barrier gene. In some embodiments, the expression level of the at least one lineage-determining factor is increased within 48 hours after reducing the expression level(s) of the at least one barrier gene. In some embodiments, the expression level of the at least one lineage-determining factor is increased within 42 hours after reducing the expression level(s) of the at least one barrier gene. In some embodiments, the expression level of the at least one lineage-determining factor is increased within 36 hours after reducing the expression level(s) of the at least one barrier gene. In some embodiments, the expression level of the at least one lineage-determining factor is increased within 30 hours after reducing the expression level(s) of the at least one barrier gene. In some embodiments, the expression level of the at least one lineage-determining factor is increased within 24 hours after reducing the expression level(s) of the at least one barrier gene. In some embodiments, the expression level of the at least one lineage-determining factor is increased within 18 hours after reducing the expression level(s) of the at least one barrier gene. In some embodiments, the expression level of the at least one lineage-determining factor is increased within 12 hours after reducing the expression level(s) of the at least one barrier gene. In some embodiments, the expression level of the at least one lineage-determining factor is increased within 6 hours after reducing the expression level(s) of the at least one barrier gene. In some embodiments, the expression level of the at least one lineage-determining factor is increased within 3 hours after reducing the expression level(s) of the at least one barrier gene. In some embodiments, the expression level of the at least one lineage-determining factor is increased within 1 hour after reducing the expression level(s) of the at least one barrier gene.

In some embodiments, the expression level of the at least one lineage-determining factor is increased before reducing the expression level(s) of the at least one barrier gene. In some embodiments, the expression level of the at least one lineage-determining factor is increased within 10 days before reducing the expression level(s) of the at least one barrier gene. In some embodiments, the expression level of the at least one lineage-determining factor is increased within 9 days before reducing the expression level(s) of the at least one barrier gene. In some embodiments, the expression level of the at least one lineage-determining factor is increased within 8 days before reducing the expression level(s) of the at least one barrier gene. In some embodiments, the expression level of the at least one lineage-determining factor is increased within 7 days before reducing the expression level(s) of the at least one barrier gene. In some embodiments, the expression level of the at least one lineage-determining factor is increased within 6 days before reducing the expression level(s) of the at least one barrier gene. In some embodiments, the expression level of the at least one lineage-determining factor is increased within 5 days before reducing the expression level(s) of the at least one barrier gene. In some embodiments, the expression level of the at least one lineage-determining factor is increased within 4 days before reducing the expression level(s) of the at least one barrier gene. In some embodiments, the expression level of the at least one lineage-determining factor is increased within 72 hours before reducing the expression level(s) of the at least one barrier gene. In some embodiments, the expression level of the at least one lineage-determining factor is increased within 60 hours before reducing the expression level(s) of the at least one barrier gene. In some embodiments, the expression level of the at least one lineage-determining factor is increased within 48 hours before reducing the expression level(s) of the at least one barrier gene. In some embodiments, the expression level of the at least one lineage-determining factor is increased within 42 hours before reducing the expression level(s) of the at least one barrier gene. In some embodiments, the expression level of the at least one lineage-determining factor is increased within 36 hours before reducing the expression level(s) of the at least one barrier gene. In some embodiments, the expression level of the at least one lineage-determining factor is increased within 30 hours before reducing the expression level(s) of the at least one barrier gene. In some embodiments, the expression level of the at least one lineage-determining factor is increased within 24 hours before reducing the expression level(s) of the at least one barrier gene. In some embodiments, the expression level of the at least one lineage-determining factor is increased within 18 hours before reducing the expression level(s) of the at least one barrier gene. In some embodiments, the expression level of the at least one lineage-determining factor is increased within 12 hours before reducing the expression level(s) of the at least one barrier gene. In some embodiments, the expression level of the at least one lineage-determining factor is increased within 6 hours before reducing the expression level(s) of the at least one barrier gene. In some embodiments, the expression level of the at least one lineage-determining factor is increased within 3 hours before reducing the expression level(s) of the at least one barrier gene. In some embodiments, the expression level of the at least one lineage-determining factor is increased within 1 hour before reducing the expression level(s) of the at least one barrier gene.

In some embodiments, the expression level of the at least one lineage-determining factor is increased simultanousely with reducing the expression level(s) of the at least one barrier gene.

In some embodiments, the expression level of the at least one lineage-determining factor may be increased by inhibiting a suppressor upstream of the at least one lineage-determining factor. In some embodiments, the suppressor may be inhibited by at least one antisense oligonucleotide targeting the suppressor. In some embodiments, the at least one antisense oligonucleotide is an antisense small noncoding RNA (sRNA). In some embodiments, the suppressor may be inhibited by at least one small interfering RNA (siRNA) targeting the suppressor. In some embodiments, the suppressor may be inhibited by a gene editing tool. In some embodiments, the gene editing tool is CRISPR/Cas9. In some embodiments, the suppressor may be inhibited by a small molecule targeting the suppressor. In some embodiments, the expression level of the at least one lineage-determining factor may be increased by overexpression the at least one lineage-determining factor in a cell. In some embodiments, the overexpression of the at least one lineage-determining factor in the cell may be achieved by transfecting the cell with at least one vector carrying the at least one lineage-determining factor. In some embodiments, the at least one vector is a virus. The virus may be a lentivirus or an adeno-associated virus.

In some embodiments, the at least one lineage-determining factor is selected from the group consisting of MEF2C, GATA4, TBX5, ASCL1, BRN2, MYTL1, OCT4, KLF4, SOX2, MYC and MYOD. In some embodiments, the at least one lineage-determining factor is MEF2C. In some embodiments, the at least one lineage-determining factor is GATA4. In some embodiments, the at least one lineage-determining factor is TBX5. In some embodiments, the at least one lineage-determining factor is ASCL1. In some embodiments, the at least one lineage-determining factor is BRN2. In some embodiments, the at least one lineage-determining factor is MYTL1. In some embodiments, the at least one lineage-determining factor is OCT4. In some embodiments, the at least one lineage-determining factor is KLF4. In some embodiments, the at least one lineage-determining factor is SOX2. In some embodiments, the at least one lineage-determining factor is MYC. In some embodiments, the at least one lineage-determining factor is MYOD. In some embodiments, the at least one lineage-determining factor comprises or consists ofASCL1, BRN2, and MYTL1. In some embodiments, the at least one lineage-determining factor comprises or consists of MEF2C, GATA4, and TBX5. In some embodiments, the at least one lineage-determining factor comprises or consists of OCT4, KLF4, SOX2, and MYC.

In some embodiments, the cell of a different cell type may be any cell type. In some embodiments, the cell of a different cell type may be a somatic cell. In some embodiments, the cell of a different cell type may be epithelia cells, nerve cells, muscle cells, or connective tissue cells. In some embodiments, the cell of a different cell type may be a differentiated cell. In some embodiments, the differentiated cell may be completely differentiated or partially differentiated. In some embodiments, the cell of a different cell type is a fibroblast, an endothelial cell, or a peripheral blood mononuclear cell (PBMC).

In some embodiments, the method further comprises altering chromatin structure of the cell of a different cell type compared to a wild-type cell of the cell of a different cell type. In some embodiments, the altering chromatin structure of the cell of a different cell type compared to a wild-type cell of the cell of a different cell type comprises altering the direct binding pattern of the at least one barrier gene to the chromatin. In some embodiments, the altering chromatin structure of the cell of a different cell type compared to a wild-type cell of the cell of a different cell type comprises altering the pattern of the at least one barrier gene binding to AP-1 motif-enriched open chromatin scattered throughout the genome, thereby limiting the at least one LDF's ability to remodel the chromatin and promote cell fate reprogramming. In some embodiments, the altering chromatin structure of the cell of a different cell type compared to a wild-type cell of the cell of a different cell type comprises altering the pattern of the at least one barrier gene binding to chromatin via prototypical AP-1 (TRE: TGACTCA) motifs. In some embodiments, the altering chromatin structure of the cell of a different cell type compared to a wild-type cell of the cell of a different cell type comprises altering the pattern of the at least one barrier gene binding to chromatin via partial AP-1 motifs (CRE: TGACGT-).

In some embodiments, the cell of a different cell type has a reduced number of motifs bound to a protein expressed by the at least one barrier gene compared to a wild-type cell of the cell of a different cell type. In some embodiments, the number of the protein expressed by the at least one barrier gene to the chromatin is reduced. In some embodiments, the number of the protein expressed by the at least one barrier gene that binds to AP-1 motif-enriched open chromatin is reduced. In some embodiments, the number of the binding to the chromatin by the protein expressed by the at least one barrier gene is reduced. In some embodiments, the at least one barrier gene is ATF7IP, JUNB, SP7, or ZNF207.

In some embodiments, the cell of interest may be any cell type. In some embodiments, the cell of interest may be a somatic cell. In some embodiments, the cell of interest may be epithelia cells, nerve cells, muscle cells, or connective tissue cells. In some embodiments, the cell of interest may be completely differentiated or partially differentiated. In some embodiments, the cell of interest may be a cardiomyocyte-like cell, a cardiomyocyte, a neuron, a skeletal muscle cell, or an induced pluripotent stem cell. In some embodiments, the method further comprises coverting the cell of a different cell type into an induced pluripotent stem cell (iPSC) state.

In some embodiments, the cell of interest has an increased expression of at least one differentiation marker of a specific cell lineage as compared to the cell of a different cell type. In some embodiments, the differentiation marker may be at least one cardiac marker. In some embodiments, the at least one cardiac marker is selected from the group consisting of ACTC1, MYL7, TNNT2, SCN5A, RYR2, NPPA, and NPPB. In some embodiments, the differentiation marker may be at least one neuron-specific marker. In some embodiments, the at least one neuron-specific marker is selected from the group consisting of vGLUT2, GAD67, PVALB, and SYN1. In some embodiments, the cell of interest has an increased expression of at least one stem cell marker as compared to the cell of a different cell type. In some embodiments, the at least one stem cell marker is selected from the group consisting of SSEA4 and NANOG.

### Methods to Reduce the Expression Level(s) of Barrier Genes

The expression level(s) of the at least one barrier gene can be reduced by using any gene editing tool known by the skilled in the art. In some embodiments, the expression level(s) of at least one barrier gene is reduced by contacting the cell of a different cell type with at least one gene editing tool targeting the at least one barrier gene. In some embodiments, the gene editing tool is CRISPR/Cas9. In some cases, a gene editing tool or system can be complexed with a guide polynucleotide that is complementary to a barrier gene disclosed herein or a portion thereof. Further, in some cases, a gRNA or gDNA comprises a sequence that binds a target sequence within or adjacent to a barrier gene disclosd herein. In some cases, a guide polynucleotide binds a portion of a barrier gene disclosed herein. A target sequence can contain mismatchs and still allow for binding and functionality of a gene editing tool or system. In some embodiments, the gene editing tool is a TALE nuclease (TALEN), transposon-based nuclease, argonaute, sleeping beauty, ZEN, meganuclease, or Mega-TAL. In some embodiments, the gene editing tool is an antisense oligonucleotide. In some embodiments, the antisense oligonucleotide is an antisense small noncoding RNA (sRNA). In some embodiments, the sRNA is a microRNA (miRNA) or a short interfering RNA (siRNA). In some embodiments, the expression level(s) of at least one barrier gene is reduced by contacting the cell of a different cell type with at least one siRNA molecule targeting said at least one barrier gene. In some embodiments, the expression level(s) of at least one barrier gene is reduced by contacting the cell of a different cell type with at least one small molecule targeting the at least one barrier gene or a gene product thereof (e.g., mRNA or protein). In some embodiments, the expression level(s) of at least one barrier gene is reduced by contacting the cell of a different cell type with a gene editing tool targeting a gene of a regulatory protein regulating the expression level(s) of the at least one barrier gene. In some embodiments, the expression level(s) of at least one barrier gene is reduced by contacting the cell of a different cell type with a small molecule targeting a regulatory protein regulating the expression level(s) of the at least one barrier gene.

### Pharmaceutical Composition

In another aspect, the present disclosure provides pharmaceutical compositions, comprising at least one inhibitor of at least one barrier gene selected from the group consisting of ATF7IP, JUNB, ZNF207, Sp7, FOXA1, HEXIM2, SMARCA5, SOX15, CHST2 and NCEH1, and a pharmaceutically acceptable carrier or excipient; wherein when said at least one barrier gene is ATF7IP or SOX15, at least a second inhibitor of a second barrier gene is included. In some embodiments, the at least one barrier gene comprises ATF7IP and a second barrier gene selected from the group consisting of JUNB, ZNF207, Sp7, FOXA1, HEXIM2, SMARCA5, SOX15, CHST2 and NCEH1. In some embodiments, the at least one barrier gene comprises JUNB and a second barrier gene that is selected from the group consisting of ATF7IP, ZNF207, Sp7, FOXA1, HEXIM2, SMARCA5, SOX15, CHST2 and NCEH1. In some embodiments, the at least one barrier gene comprises ZNF207 and a second barrier gene that is selected from the group consisting of ATF7IP, JUNB, Sp7, FOXA1, HEXIM2, SMARCA5, SOX15, CHST2 and NCEH1. In some embodiments, the at least one barrier gene comprises Sp7 and a second barrier gene that is selected from the group consisting of ATF7IP, JUNB, ZNF207, FOXA1, HEXIM2, SMARCA5, SOX15, CHST2 and NCEH1. In some embodiments, the at least one barrier gene comprises FOXA1 and a second barrier gene that is selected from the group consisting of ATF7IP, JUNB, ZNF207, Sp7, HEXIM2, SMARCA5, SOX15, CHST2 and NCEH1. In some embodiments, the at least one barrier gene comprises HEXIM2 and a second barrier gene that is selected from the group consisting of ATF7IP, JUNB, ZNF207, Sp7, FOXA1, SMARCA5, SOX15, CHST2 and NCEH1. In some embodiments, the at least one barrier gene comprises SMARCA5 and a second barrier gene that is selected from the group consisting of ATF7IP, JUNB, ZNF207, Sp7, FOXA1, HEXIM2, SOX15, CHST2 and NCEH1. In some embodiments, the at least one barrier gene comprises SOX15 and a second barrier gene that is selected from the group consisting of ATF7IP, JUNB, ZNF207, Sp7, FOXA1, HEXIM2, SMARCA5, CHST2 and NCEH1. In some embodiments, the at least one barrier gene comprises CHST2 and a second barrier gene that is selected from the group consisting of ATF7IP, JUNB, ZNF207, Sp7, FOXA1, HEXIM2, SMARCA5, SOX15 and NCEH1. In some embodiments, the at least one barrier gene comprises NCEH1 and a second barrier gene that is selected from the group consisting of ATF7IP, JUNB, ZNF207, Sp7, FOXA1, HEXIM2, SMARCA5, SOX15 and CHST2. In some embodiments, the at least one barrier gene comprises ATF7IP and JUNB. In some embodiments, the at least one barrier gene comprises ATF7IP and ZNF207. In some embodiments, the at least one barrier gene comprises ATF7IP and Sp7. In some embodiments, the at least one barrier gene comprises ATF7IP and FOXA1. In some embodiments, the at least one barrier gene comprises ATF7IP and HEXIM2. In some embodiments, the at least one barrier gene comprises ATF7IP and SMARCA5. In some embodiments, the at least one barrier gene comprises ATF7IP and SOX15. In some embodiments, the at least one barrier gene comprises ATF7IP and CHST2. In some embodiments, the at least one barrier gene comprises ATF7IP and NCEH1. In some embodiments, the at least one barrier gene comprises JUNB and ZNF207. In some embodiments, the at least one barrier gene comprises JUNB and Sp7. In some embodiments, the at least one barrier gene comprises JUNB and FOXA1. In some embodiments, the at least one barrier gene comprises JUNB and HEXIM2. In some embodiments, the at least one barrier gene comprises JUNB and SMARCA5. In some embodiments, the at least one barrier gene comprises JUNB and SOX15. In some embodiments, the at least one barrier gene comprises JUNB and CHST2. In some embodiments, the at least one barrier gene comprises JUNB and NCEH1. In some embodiments, the at least one barrier gene comprises ZNF207 and Sp7. In some embodiments, the at least one barrier gene comprises ZNF207 and FOXA1. In some embodiments, the at least one barrier gene comprises ZNF207 and HEXIM2. In some embodiments, the at least one barrier gene comprises ZNF207 and SMARCA5. In some embodiments, the at least one barrier gene comprises ZNF207 and SOX15. In some embodiments, the at least one barrier gene comprises ZNF207 and CHST2. In some embodiments, the at least one barrier gene comprises ZNF207 and NCEH1. In some embodiments, the at least one barrier gene comprises Sp7 and FOXA1. In some embodiments, the at least one barrier gene comprises Sp7 and HEXIM2. In some embodiments, the at least one barrier gene comprises Sp7 and SMARCA5. In some embodiments, the at least one barrier gene comprises Sp7 and SOX15. In some embodiments, the at least one barrier gene comprises Sp7 and CHST2. In some embodiments, the at least one barrier gene comprises Sp7 and NCEH1. In some embodiments, the at least one barrier gene comprises FOXA1 and HEXIM. In some embodiments, the at least one barrier gene comprises FOXA1 and SMARCA5. In some embodiments, the at least one barrier gene comprises FOXA1 and SOX15. In some embodiments, the at least one barrier gene comprises FOXA1 and CHST2. In some embodiments, the at least one barrier gene comprises FOXA1 and NCEH1.

In some embodiments, the at least one barrier gene comprises HEXIM2 and SMARCA5. In some embodiments, the at least one barrier gene comprises HEXIM2 and SOX15. In some embodiments, the at least one barrier gene comprises HEXIM2 and CHST2. In some embodiments, the at least one barrier gene comprises HEXIM2 and NCEH1. In some embodiments, the at least one barrier gene comprises SMARCA5 and SOX15. In some embodiments, the at least one barrier gene comprises SMARCA5 and CHST2. In some embodiments, the at least one barrier gene comprises SMARCA5 and NCEH1. In some embodiments, the at least one barrier gene comprises SOX15 and CHST2. In some embodiments, the at least one barrier gene comprises SOX15 and NCEH1. In some embodiments, the at least one barrier gene comprises CHST2 and NCEH1.

In some embodiments, the at least one barrier gene comprises ATF7IP and JUNB. In some embodiments, the at least one barrier gene comprises ATF7IP and ZNF207. In some embodiments, the at least one barrier gene comprises ATF7IP and Sp7. In some embodiments, the at least one barrier gene comprises ATF7IP and FOXA1. In some embodiments, the at least one barrier gene comprises ATF7IP and HEXIM2. In some embodiments, the at least one barrier gene comprises ATF7IP and SMARCA5. In some embodiments, the at least one barrier gene comprises ATF7IP and SOX15. In some embodiments, the at least one barrier gene comprises ATF7IP and CHST2. In some embodiments, the at least one barrier gene comprises ATF7IP and NCEH1. In some embodiments, the at least one barrier gene comprises JUNB and ZNF207. In some embodiments, the at least one barrier gene comprises JUNB and Sp7. In some embodiments, the at least one barrier gene comprises JUNB and FOXA1. In some embodiments, the at least one barrier gene comprises JUNB and HEXIM2. In some embodiments, the at least one barrier gene comprises JUNB and SMARCA5. In some embodiments, the at least one barrier gene comprises JUNB and SOX15. In some embodiments, the at least one barrier gene comprises JUNB and CHST2. In some embodiments, the at least one barrier gene comprises JUNB and NCEH1. In some embodiments, the at least one barrier gene comprises ZNF207 and Sp7. In some embodiments, the at least one barrier gene comprises ZNF207 and FOXA1. In some embodiments, the at least one barrier gene comprises ZNF207 and HEXIM2. In some embodiments, the at least one barrier gene comprises ZNF207 and SMARCA5. In some embodiments, the at least one barrier gene comprises ZNF207 and SOX15. In some embodiments, the at least one barrier gene comprises ZNF207 and CHST2. In some embodiments, the at least one barrier gene comprises ZNF207 and NCEH1. In some embodiments, the at least one barrier gene comprises Sp7 and FOXA1. In some embodiments, the at least one barrier gene comprises Sp7 and HEXIM2. In some embodiments, the at least one barrier gene comprises Sp7 and SMARCA5. In some embodiments, the at least one barrier gene comprises Sp7 and SOX15. In some embodiments, the at least one barrier gene comprises Sp7 and CHST2. In some embodiments, the at least one barrier gene comprises Sp7 and NCEH1. In some embodiments, the at least one barrier gene comprises FOXA1 and HEXIM2. In some embodiments, the at least one barrier gene comprises FOXA1 and SMARCA5. In some embodiments, the at least one barrier gene comprises FOXA1 and SOX15. In some embodiments, the at least one barrier gene comprises FOXA1 and CHST2. In some embodiments, the at least one barrier gene comprises FOXA1 and NCEH1. In some embodiments, the at least one barrier gene comprises HEXIM2 and SMARCA5. In some embodiments, the at least one barrier gene comprises HEXIM2 and SOX15. In some embodiments, the at least one barrier gene comprises HEXIM2 and CHST2. In some embodiments, the at least one barrier gene comprises HEXIM2 and NCEH1. In some embodiments, the at least one barrier gene comprises SMARCA5 and SOX15. In some embodiments, the at least one barrier gene comprises SMARCA5 and CHST2. In some embodiments, the at least one barrier gene comprises SMARCA5 and NCEH1. In some embodiments, the at least one barrier gene comprises SOX15 and CHST2. In some embodiments, the at least one barrier gene comprises SOX15 and NCEH1. In some embodiments, the at least one barrier gene comprises CHST2 and NCEH1.

In some embodiments, the at least one barrier gene comprises ATF7IP, JUNB, and ZNF207. In some embodiments, the at least one barrier gene comprises ATF7IP, JUNB, and Sp7. In some embodiments, the at least one barrier gene comprises ATF7IP, JUNB, and FOXA1. In some embodiments, the at least one barrier gene comprises ATF7IP, JUNB, and HEXIM2. In some embodiments, the at least one barrier gene comprise ATF7IP, JUNB, and SMARCA5. In some embodiments, the at least one barrier gene comprises ATF7IP, JUNB, and SOX15. In some embodiments, the at least one barrier gene comprise ATF7IP, JUNB, and CHST2. In some embodiments, the at least one barrier gene comprises ATF7IP, JUNB, and NCEH1. In some embodiments, the at least one barrier gene comprises ATF7IP, ZNF207, and Sp7. In some embodiments, the at least one barrier gene comprises ATF7IP, ZNF207, and FOXA1. In some embodiments, the at least one barrier gene comprises ATF7IP, ZNF207, and HEXIM2. In some embodiments, the at least one barrier gene comprises ATF7IP, ZNF207, and SMARCA5. In some embodiments, the at least one barrier gene comprises ATF7IP, ZNF207, and SOX15. In some embodiments, the at least one barrier gene comprises ATF7IP, ZNF207, and CHST2. In some embodiments, the at least one barrier gene comprises ATF7IP, ZNF207, and NCEH1. In some embodiments, the at least one barrier gene comprises ATF7IP, Sp7, and FOXA1. In some embodiments, the at least one barrier gene comprises ATF7IP, Sp7, and HEXIM2. In some embodiments, the at least one barrier gene comprise ATF7IP, Sp7, and SMARCA5. In some embodiments, the at least one barrier gene comprises ATF7IP, Sp7, and SOX15. In some embodiments, the at least one barrier gene comprises ATF7IP, Sp7, and CHST2. In some embodiments, the at least one barrier gene comprises ATF7IP, Sp7, and NCEH1. In some embodiments, the at least one barrier gene comprises ATF7IP, FOXA1, and HEXIM2. In some embodiments, the at least one barrier gene comprises ATF7IP, FOXA1, and SMARCA5. In some embodiments, the at least one barrier gene comprises ATF7IP, FOXA1, and SOX15. In some embodiments, the at least one barrier gene comprises ATF7IP, FOXA1, and CHST2. In some embodiments, the at least one barrier gene comprises ATF7IP, FOXA1, and NCEH1. In some embodiments, the at least one barrier gene comprises ATF7IP, HEXIM2, and SMARCA5. In some embodiments, the at least one barrier gene comprises ATF7IP, HEXIM2, and SOX15. In some embodiments, the at least one barrier gene comprises ATF7IP, HEXIM2, and CHST2. In some embodiments, the at least one barrier gene comprises ATF7IP, HEXIM2, and NCEH1. In some embodiments, the at least one barrier gene comprises ATF7IP, SMARCA5, and SOX15. In some embodiments, the at least one barrier gene comprises ATF7IP, SMARCA5, and CHST2. In some embodiments, the at least one barrier gene comprises ATF7IP, SMARCA5, and NCEH1. In some embodiments, the at least one barrier gene comprises ATF7IP, SOX15, and CHST2. In some embodiments, the at least one barrier gene comprises ATF7IP, SOX15, and NCEH1. In some embodiments, the at least one barrier gene comprises JUNB, ZNF207, and Sp7. In some embodiments, the at least one barrier gene comprises JUNB, ZNF207, and FOXA1. In some embodiments, the at least one barrier gene comprises JUNB, ZNF207, and HEXIM2. In some embodiments, the at least one barrier gene comprises JUNB, ZNF207, and SMARCA5. In some embodiments, the at least one barrier gene comprises JUNB, ZNF207, and SOX15. In some embodiments, the at least one barrier gene comprises JUNB, ZNF207, and CHST2. In some embodiments, the at least one barrier gene comprises JUNB, ZNF207, and NCEH1. In some embodiments, the at least one barrier gene comprises JUNB, Sp7, and FOXA1. In some embodiments, the at least one barrier gene comprises JUNB, Sp7, and HEXIM2. In some embodiments, the at least one barrier gene comprises JUNB, Sp7, and SMARCA5. In some embodiments, the at least one barrier gene comprises JUNB, Sp7, and SOX15. In some embodiments, the at least one barrier gene comprises JUNB, Sp7, and CHST2. In some embodiments, the at least one barrier gene comprises JUNB, Sp7, and NCEH1. In some embodiments, the at least one barrier gene comprises JUNB, FOXA1, and HEXIM2. In some embodiments, the at least one barrier gene comprises JUNB, FOXA1, and SMARCA5. In some embodiments, the at least one barrier gene comprises JUNB, FOXA1, and SOX15. In some embodiments, the at least one barrier gene comprises JUNB, FOXA1, and CHST2. In some embodiments, the at least one barrier gene comprises JUNB, FOXA1, and NCEH1. In some embodiments, the at least one barrier gene comprises JUNB, HEXIM2, and SMARCA5. In some embodiments, the at least one barrier gene comprises JUNB, HEXIM2, and SOX15. In some embodiments, the at least one barrier gene comprises JUNB, HEXIM2, and CHST2. In some embodiments, the at least one barrier gene comprises JUNB, HEXIM2, and NCEH1. In some embodiments, the at least one barrier gene comprises JUNB, SMARCA5, and SOX15. In some embodiments, the at least one barrier gene comprises JUNB, SMARCA5, and CHST2. In some embodiments, the at least one barrier gene comprises JUNB, SMARCA5, and NCEH1. In some embodiments, the at least one barrier gene comprises JUNB, SOX15, and CHST2. In some embodiments, the at least one barrier gene comprises JUNB, SOX15, and NCEH1. In some embodiments, the at least one barrier gene comprises JUNB, CHST2 and NCEH1. In some embodiments, the at least one barrier gene comprises ZNF207, Sp7, and FOXA1. In some embodiments, the at least one barrier gene comprises ZNF207, Sp7, and HEXIM2. In some embodiments, the at least one barrier gene comprises ZNF207, Sp7, and SMARCA5. In some embodiments, the at least one barrier gene comprises ZNF207, Sp7, and SOX15. In some embodiments, the at least one barrier gene comprises ZNF207, Sp7, and CHST2. In some embodiments, the at least one barrier gene comprises ZNF207, Sp7, and NCEH1. In some embodiments, the at least one barrier gene comprises ZNF207, FOXA1, and HEXIM2. In some embodiments, the at least one barrier gene comprises ZNF207, FOXA1, and SMARCA5. In some embodiments, the at least one barrier gene comprises ZNF207, FOXA1, and SOX15. In some embodiments, the at least one barrier gene comprises ZNF207, FOXA1, and CHST2. In some embodiments, the at least one barrier gene comprises ZNF207, FOXA1, and NCEH1. In some embodiments, the at least one barrier gene comprises ZNF207, HEXIM2, and SMARCA5. In some embodiments, the at least one barrier gene comprises ZNF207, HEXIM2, and SOX15. In some embodiments, the at least one barrier gene comprises ZNF207, HEXIM2, and CHST2. In some embodiments, the at least one barrier gene comprises ZNF207, HEXIM2, and NCEH1. In some embodiments, the at least one barrier gene comprises ZNF207, SMARCA5, and SOX15. In some embodiments, the at least one barrier gene comprises ZNF207, SMARCA5, and CHST2. In some embodiments, the at least one barrier gene comprises ZNF207, SMARCA5, and NCEH1. In some embodiments, the at least one barrier gene comprises ZNF207, SOX15, and CHST2. In some embodiments, the at least one barrier gene comprises ZNF207, SOX15, and NCEH1. In some embodiments, the at least one barrier gene comprises ZNF207, CHST2 and NCEH1. In some embodiments, the at least one barrier gene comprises ZNF207, HEXIM2, and SMARCA5. In some embodiments, the at least one barrier gene comprises ZNF207, HEXIM2, and SOX15. In some embodiments, the at least one barrier gene comprises ZNF207, HEXIM2, and CHST2. In some embodiments, the at least one barrier gene comprises ZNF207, HEXIM2, and NCEH1. In some embodiments, the at least one barrier gene comprises ZNF207, SMARCA5, and SOX15. In some embodiments, the at least one barrier gene comprises ZNF207, SMARCA5, and CHST2. In some embodiments, the at least one barrier gene comprises ZNF207, SMARCA5, and NCEH1. In some embodiments, the at least one barrier gene comprises ZNF207, SOX15, and CHST2. In some embodiments, the at least one barrier gene comprises ZNF207, SOX15, and NCEH1. In some embodiments, the at least one barrier gene comprises ZNF207, CHST2 and NCEH1. In some embodiments, the at least one barrier gene comprises Sp7, FOXA1, and HEXIM2. In some embodiments, the at least one barrier gene comprises Sp7, FOXA1, and SMARCA5. In some embodiments, the at least one barrier gene comprises Sp7, FOXA1, and SOX15. In some embodiments, the at least one barrier gene comprises Sp7, FOXA1, and CHST2. In some embodiments, the at least one barrier gene comprises Sp7, FOXA1, and NCEH1. In some embodiments, the at least one barrier gene comprises FOXA1, HEXIM2, and SMARCA5. In some embodiments, the at least one barrier gene comprises FOXA1, HEXIM2, and SOX15. In some embodiments, the at least one barrier gene comprises FOXA1, HEXIM2, and CHST2. In some embodiments, the at least one barrier gene comprises FOXA1, HEXIM2, and NCEH1. In some embodiments, the at least one barrier gene comprises HEXIM2, SMARCA5, and SOX15. In some embodiments, the at least one barrier gene comprises HEXIM2, SMARCA5, and CHST2. In some embodiments, the at least one barrier gene comprises HEXIM2, SMARCA5, and NCEH1. In some embodiments, the at least one barrier gene comprises SMARCA5, SOX15, and CHST2. In some embodiments, the at least one barrier gene comprises SMARCA5, SOX15, and NCEH1. In some embodiments, the at least one barrier gene comprises SOX15, CHST2, and NCEH1.

In another aspect, the present disclosure provides pharmaceutical compositions, comprising at least one inhibitor of at least two barrier genes selected from the group consisting of ATF7IP, JUNB, ZNF207, and Sp7, and a pharmaceutically acceptable carrier or excipient. In some embodiments, the at least two barrier genes comprise ATF7IP and JUNB. In some embodiments, the at least two barrier genes comprise ATF7IP and ZNF207. In some embodiments, the at least two barrier genes comprise ATF7IP and Sp7. In some embodiments, the at least two barrier genes comprise JUNB and ZNF207. In some embodiments, the at least two barrier genes comprise JUNB and Sp7. In some embodiments, the at least two barrier genes comprise ZNF207 and Sp7.

In some embodiments, the at least one barrier gene comprises ATF7IP, JUNB, and a third barrier gene that is selected from the group consisting of ZNF207, Sp7, FOXA1, HEXIM2, SMARCA5, SOX15, CHST2 and NCEH1. In some embodiments, the at least two barrier gene comprise ATF7IP, JUNB, and ZNF207. In some embodiments, the at least two barrier gene comprise ATF7IP, JUNB, and Sp7. In some embodiments, the at least two barrier gene comprise ATF7IP, JUNB, and FOXA1. In some embodiments, the at least two barrier gene comprise ATF7IP, JUNB, and HEXIM2. In some embodiments, the at least two barrier gene comprise ATF7IP, JUNB, and SMARCA5. In some embodiments, the at least two barrier gene comprise ATF7IP, JUNB, and SOX15. In some embodiments, the at least two barrier gene comprise ATF7IP, JUNB, and CHST2. In some embodiments, the at least two barrier gene comprise ATF7IP, JUNB, and NCEH1.

In some embodiments, the at least one barrier gene comprises ATF7IP, ZNF207, and a third barrier gene that is selected from the group consisting of JUNB, Sp7, FOXA1, HEXIM2, SMARCA5, SOX15, CHST2 and NCEH1. In some embodiments, the at least two barrier genes comprise ATF7IP, ZNF207, and JUNB. In some embodiments, the at least two barrier genes comprise ATF7IP, ZNF207, and Sp7. In some embodiments, the at least two barrier genes comprise ATF7IP, ZNF207, and FOXA1. In some embodiments, the at least two barrier genes comprise ATF7IP, ZNF207, and HEXIM2. In some embodiments, the at least two barrier genes comprise ATF7IP, ZNF207, and SMARCA5. In some embodiments, the at least two barrier genes comprise ATF7IP, ZNF207, and SOX15. In some embodiments, the at least two barrier genes comprise ATF7IP, ZNF207, and CHST2. In some embodiments, the at least two barrier genes comprise ATF7IP, ZNF207, and NCEH1.

In some embodiments, the at least one barrier gene comprises ATF7IP, Sp7, and a third barrier gene that is selected from the group consisting of JUNB, ZNF207, FOXA1, HEXIM2, SMARCA5, SOX15, CHST2 and NCEH1. In some embodiments, the at least two barrier genes comprise ATF7IP, Sp7, and JUNB. In some embodiments, the at least two barrier genes comprise ATF7IP, Sp7, and ZNF207. In some embodiments, the at least two barrier genes comprise ATF7IP, Sp7, and FOXA1. In some embodiments, the at least two barrier genes comprise ATF7IP, Sp7, and HEXIM2. In some embodiments, the at least two barrier genes comprise ATF7IP, Sp7, and SMARCA5. In some embodiments, the at least two barrier genes comprise ATF7IP, Sp7, and SOX15. In some embodiments, the at least two barrier genes comprise ATF7IP, Sp7, and CHST2. In some embodiments, the at least two barrier genes comprise ATF7IP, Sp7, and NCEH1.

In some embodiments, the at least one barrier gene comprises JUNB, ZNF207, and a third barrier gene that is selected from the group consisting of ATF7IP, Sp7, FOXA1, HEXIM2, SMARCA5, SOX15, CHST2 and NCEH1. In some embodiments, the at least two barrier genes comprise JUNB, ZNF207, and ATF7IP. In some embodiments, the at least two barrier genes comprise JUNB, ZNF207, and Sp7. In some embodiments, the at least two barrier genes comprise JUNB, ZNF207, and FOXA1. In some embodiments, the at least two barrier genes comprise JUNB, ZNF207, and HEXIM2. In some embodiments, the at least two barrier genes comprise JUNB, ZNF207, and SMARCA5. In some embodiments, the at least two barrier genes comprise JUNB, ZNF207, and SOX15. In some embodiments, the at least two barrier genes comprise JUNB, ZNF207, and CHST2. In some embodiments, the at least two barrier genes comprise JUNB, ZNF207, and NCEH1.

In some embodiments, the at least one barrier gene comprises JUNB, Sp7, and a third barrier gene that is selected from the group consisting of ATF7IP, ZNF207, FOXA1, HEXIM2, SMARCA5, SOX15, CHST2 and NCEH1. In some embodiments, the at least two barrier genes comprise JUNB, Sp7, and ATF7IP. In some embodiments, the at least two barrier genes comprise JUNB, Sp7, and ZNF207. In some embodiments, the at least two barrier genes comprise JUNB, Sp7, and FOXA1. In some embodiments, the at least two barrier genes comprise JUNB, Sp7, and HEXIM2. In some embodiments, the at least two barrier genes comprise JUNB, Sp7, and SMARCA5. In some embodiments, the at least two barrier genes comprise JUNB, Sp7, and SOX15. In some embodiments, the at least two barrier genes comprise JUNB, Sp7, and CHST2. In some embodiments, the at least two barrier genes comprise JUNB, Sp7, and NCEH1.

In some embodiments, the at least one barrier gene comprises ZNF207, Sp7, and a third barrier gene that is selected from the group consisting of ATF7IP, JUNB, FOXA1, HEXIM2, SMARCA5, SOX15, CHST2 and NCEH1. In some embodiments, the at least two barrier genes comprise ZNF207, Sp7, and ATF7IP. In some embodiments, the at least two barrier genes comprise ZNF207, Sp7, and JUNB. In some embodiments, the at least two barrier genes comprise ZNF207, Sp7, and FOXA1. In some embodiments, the at least two barrier genes comprise ZNF207, Sp7, and HEXIM2. In some embodiments, the at least two barrier genes comprise ZNF207, Sp7, and SMARCA5. In some embodiments, the at least two barrier genes comprise ZNF207, Sp7, and SOX15. In some embodiments, the at least two barrier genes comprise ZNF207, Sp7, and CHST2. In some embodiments, the at least two barrier genes comprise ZNF207, Sp7, and NCEH1.

In some embodiments, the at least two barrier genes comprise ATF7IP and JUNB. In some embodiments, the at least two barrier genes comprise ATF7IP and ZNF207. In some embodiments, the at least two barrier genes comprise ATF7IP and Sp7. In some embodiments, the at least two barrier genes comprise JUNB and ZNF207. In some embodiments, the at least two barrier genes comprise JUNB and Sp7. In some embodiments, the at least two barrier genes comprise ZNF207 and Sp7. In some embodiments, the at least one barrier gene comprises ATF7IP, JUNB, and ZNF207. In some embodiments, the at least one barrier gene comprises ATF7IP, JUNB, and Sp7. In some embodiments, the at least one barrier gene comprises JUNB, ZNF207, and Sp7. In some embodiments, the at least one barrier gene comprises ATF7IP, ZNF207, and Sp7.

In some embodiments, the at least two barrier genes comprise ATF7IP, JUNB, and ZNF207 and a fourth barrier gene that is selected from the group consisting of Sp7, FOXA1, HEXIM2, SMARCA5, SOX15, CHST2 and NCEH1. In some embodiments, the at least two barrier genes comprise ATF7IP, JUNB, ZNF207 and Sp7. In some embodiments, the at least two barrier genes comprise ATF7IP, JUNB, ZNF207 and FOXA1. In some embodiments, the at least two barrier genes comprise ATF7IP, JUNB, ZNF207 and HEXIM2. In some embodiments, the at least two barrier genes comprise ATF7IP, JUNB, ZNF207 and SMARCA5. In some embodiments, the at least two barrier genes comprise ATF7IP, JUNB, ZNF207 and SOX15. In some embodiments, the at least two barrier genes comprise ATF7IP, JUNB, ZNF207 and CHST2. In some embodiments, the at least two barrier genes comprise ATF7IP, JUNB, ZNF207 and NCEH1.

In some embodiments, the at least two barrier genes comprise ATF7IP, JUNB, and Sp7, and a fourth barrier gene that is selected from the group consisting of ZNF207, FOXA1, HEXIM2, SMARCA5, SOX15, CHST2 and NCEH1. In some embodiments, the at least two barrier genes comprise ATF7IP, JUNB, Sp7, and ZNF207. In some embodiments, the at least two barrier genes comprise ATF7IP, JUNB, Sp7, and FOXA1. In some embodiments, the at least two barrier genes comprise ATF7IP, JUNB, Sp7, and HEXIM2. In some embodiments, the at least two barrier genes comprise ATF7IP, JUNB, Sp7, and SMARCA5. In some embodiments, the at least two barrier genes comprise ATF7IP, JUNB, Sp7, and SOX15. In some embodiments, the at least two barrier genes comprise ATF7IP, JUNB, Sp7, and CHST2. In some embodiments, the at least two barrier genes comprise ATF7IP, JUNB, Sp7, and NCEH1.

In some embodiments, the at least two barrier genes comprise JUNB, ZNF207, and Sp7, and a fourth barrier gene that is selected from the group consisting of ATF7IP, FOXA1, HEXIM2, SMARCA5, SOX15, CHST2 and NCEH1. In some embodiments, the at least two barrier genes comprise JUNB, ZNF207, Sp7, and ATF7IP. In some embodiments, the at least two barrier genes comprise JUNB, ZNF207, Sp7, and FOXA1. In some embodiments, the at least two barrier genes comprise JUNB, ZNF207, Sp7, and HEXIM2. In some embodiments, the at least two barrier genes comprise JUNB, ZNF207, Sp7, and SMARCA5. In some embodiments, the at least two barrier genes comprise JUNB, ZNF207, Sp7, and SOX15. In some embodiments, the at least two barrier genes comprise JUNB, ZNF207, Sp7, and CHST2. In some embodiments, the at least two barrier genes comprise JUNB, ZNF207, Sp7, and NCEH1.

In some embodiments, the at least two barrier genes comprise ATF7IP, ZNF207, and Sp7, and a fourth barrier gene that is selected from the group consisting of JUNB, FOXA1, HEXIM2, SMARCA5, SOX15, CHST2 and NCEH1. In some embodiments, the at least two barrier genes comprise ATF7IP, ZNF207, Sp7, and JUNB. In some embodiments, the at least two barrier genes comprise ATF7IP, ZNF207, Sp7, and FOXA1. In some embodiments, the at least two barrier genes comprise ATF7IP, ZNF207, Sp7, and HEXIM2. In some embodiments, the at least two barrier genes comprise ATF7IP, ZNF207, Sp7, and SMARCA5. In some embodiments, the at least two barrier genes comprise ATF7IP, ZNF207, Sp7, and SOX15. In some embodiments, the at least two barrier genes comprise ATF7IP, ZNF207, Sp7, and CHST2. In some embodiments, the at least two barrier genes comprise ATF7IP, ZNF207, Sp7, and NCEH1.

In some embodiments, the at least one barrier gene comprises ATF7IP, JUNB, ZNF207 and Sp7. In some embodiments, the at least two barrier genes comprise ATF7IP, JUNB, ZNF207 and Sp7 and a fifth barrier gene that is selected from the group consisting of FOXA1, HEXIM2, SMARCA5, SOX15, CHST2 and NCEH1. In some embodiments, the at least two barrier genes comprise ATF7IP, JUNB, ZNF207, Sp7, and FOXA1. In some embodiments, the at least two barrier genes comprise ATF7IP, JUNB, ZNF207, Sp7, and HEXIM2. In some embodiments, the at least two barrier genes comprise ATF7IP, JUNB, ZNF207, Sp7, and SMARCA5. In some embodiments, the at least two barrier genes comprise ATF7IP, JUNB, ZNF207, Sp7, and SOX15. In some embodiments, the at least two barrier genes comprise ATF7IP, JUNB, ZNF207, Sp7, and CHST2. In some embodiments, the at least two barrier genes comprise ATF7IP, JUNB, ZNF207, Sp7, and NCEH1.

In some embodiments, the at least two barrier genes consist of ATF7IP, JUNB, and ZNF207. In some embodiments, the at least one barrier gene consists of ATF7IP, JUNB, ZNF207 and Sp7.

In some embodiments, the at least one inhibitor comprises at least one antisense oligonucleotide targeting the at least one barrier gene. In some embodiments, the at least one antisense oligonucleotide is an antisense small noncoding RNA (sRNA). In some embodiments, the sRNA is a microRNA (miRNA) or a short interfering RNA (siRNA). In some embodiments, the at least one inhibitor comprises at least one siRNA molecule targeting said at least one barrier gene. In some embodiments, the at least one inhibitor is at least one gene editing tool targeting the at least one barrier gene. In some embodiments, the gene editing tool is CRISPR/Cas9. In some embodiments, the at least one inhibitor is at least one small molecule targeting the the at least one barrier gene or a gene product thereof (e.g., mRNA or protein).

Non-limiting examples of the pharmaceutically acceptable carrier include lipids, sugars, amino acids, lactose, glucose, fructose, sucrose, raffinose, mannose, dextrose, trehalose, trileucine, leucine, mannitol, maltitol, xylitol, glycine, sorbitol, erythritol, phosphatidylcholines (e.g., DSPC, DSPE etc.), calcium salts (e.g., calcium chloride, calcium sulfate), iron salts, starches, carbohydrates, cyclodextrins, cellulose, polyoxyethylene sorbitan fatty acid esters and derivatives, thereof.

Non-limiting examples of the pharmaceutically acceptable excipients include lipids, sugars, amino acids, lactose, glucose, fructose, sucrose, raffinose, mannose, dextrose, trehalose, trileucine, leucine, methionine, mannitol, maltitol, xylitol, glycine, sorbitol, erythritol, phosphatidylcholines (e.g., DSPC, DSPE etc.), starches, carbohydrates, cyclodextrins, calcium salts (e.g., calcium chloride, calcium sulfate), iron salts, cellulose, polyoxyethylene sorbitan fatty acid esters and derivatives, thereof.

In another aspect, the present disclosure provides a pharmaceutical compsotion comprising a first plurality of cells having a first cell type and media; wherein the pharmaceutical composition is made by a process comprising: (i) obtaining a second plurality of cells of a different cell type compared to the first cell type; (ii) reducing expression level(s) of at least one barrier gene selected from the group consisting of ATF7IP, JUNB, ZNF207, Sp7, FOXA1, HEXIM2, SMARCA5, SOX15, CHST2 and NCEH1 in the second plurality of cells; wherein when said at least one barrier gene is ATF7IP or SOX15, at least a second barrier gene is included, or reducing expression level(s) of at least two barrier genes selected from the group consisting of ATF7IP, JUNB, ZNF207, and Sp7 in the second plurality of cells.

In some embodiments, the at least one barrier gene comprises ATF7IP, JUNB, and ZNF207. In some embodiments, the at least one barrier gene further comprises Sp7. In some embodiments, the process further comprises increasing an expression level of at least one reprogramming agonist gene in the second plurality of cells. In some embodiments, the at least one reprogramming agonist gene is selected from the group consisting ofMEF2C, HSPB3, TPP1, EMC1, PPIC, IL7R, OLFML3, TCTA, and EFHD1. In some embodiments, the process further comprising increasing an expression level of at least one lineage-determining factor in the first plurality of cells. In some embodiments, the expression level of the at least one lineage-determining factor is increased within 72 hours after the expression level(s) of the at least one barrier gene is reduced. In some embodiments, the expression level of the at least one lineage-determining factor is increased within 48 hours after the expression level(s) of the at least one barrier gene is reduced. In some embodiments, the expression level of the at least one lineage-determining factor is increased within 24 hours after the expression level(s) of the at least one barrier gene is reduced. In some embodiments, the second plurality of cells is a fibroblast, an endothelial cell, or a PBMC. In some embodiments, the process further comprising altering chromatin structure of the second plurality of cells. In some embodiments, the second plurality of cells has a reduced number of motifs bound to a protein expressed by said at least one barrier gene compared to a wild-type cell of the second plurality of cells. In some embodiments, the motifs comprise an AP-1 motif. In some embodiments, the first plurality of cells is a cardiomyocyte-like cell, a cardiomyocyte, a neuron, a skeletal muscle cell, or an induced pluripotent stem cell. In some embodiments, the at least one lineage-determining factor is selected from the group consisting of MEF2C, GATA4, TBX5, ASCL1, BRN2, MYTL1, OCT4, KLF4, SOX2, MYC, and MYOD. In some embodiments, the first plurality of cells has an increased expression of at least one cardiac marker as compared to the second plurality of cells. In some embodiments, the at least one cardiac marker is selected from the group consisting of ACTC1, MYL7, TNNT2, SCN5A, RYR2, NPPA, and NPPB. In some embodiments, the first plurality of cells has an increased expression of at least one stem cell marker as compared to the second plurality of cells. In some embodiments, the at least one stem cell marker is selected from the group consisting of SSEA4 and NANOG. In some embodiments, the first plurality of cells has an increased expression of at least one neuron-specific marker as compared to the second plurality of cells. In some embodiments, the at least one neuron-specific marker is selected from the group consisting of vGLUT2, GAD67, PVALB, and SYN1. In some embodiments, the reducing expression level(s) of at least one barrier gene comprises contacting the second plurality of cells with at least one siRNA molecule targeting said at least one barrier gene. In some embodiments, the second plurality of cells are somatic cells. In some embodiments, the first plurality of cells are somatic cells. In some embodiments, the process further comprising converting the second plurality of cells into an induced pluripotent stem cell (iPSC) state.

### Tissue Regeneration

In another aspect, the present disclosure provides methods for repairing or restoring functional and structural integrity to a damaged tissue in a subject in need thereof, comprising administering to the subject an effective amount of the pharmaceutical composition as disclosed herein.

In some embodiments, the method further comprises administering at least one lineage-determining factor. In some embodiments, the at least one lineage-determining factor is administered to a subject within 10 days after administration of the pharmaceutical composition. In some embodiments, the at least one lineage-determining factor is administered to a subject within 9 days after administration of the pharmaceutical composition. In some embodiments, the at least one lineage-determining factor is administered to a subject within 8 days after administration of the pharmaceutical composition. In some embodiments, the at least one lineage-determining factor is administered to a subject within 7 days after administration of the pharmaceutical composition. In some embodiments, the at least one lineage-determining factor is administered to a subject within 6 days after administration of the pharmaceutical composition. In some embodiments, the at least one lineage-determining factor is administered to a subject within 5 days after administration of the pharmaceutical composition. In some embodiments, the at least one lineage-determining factor is administered to a subject within 4 days after administration of the pharmaceutical composition. In some embodiments, the at least one lineage-determining factor is administered to a subject within 72 hours after administration of the pharmaceutical composition. In some embodiments, the at least one lineage-determining factor is administered to a subject within 60 hours after administration of the pharmaceutical composition. In some embodiments, the at least one lineage-determining factor is administered to a subject within 48 hours after administration of the pharmaceutical composition. In some embodiments, the at least one lineage-determining factor is administered to a subject within 42 hours after administration of the pharmaceutical composition. In some embodiments, the at least one lineage-determining factor is administered to a subject within 36 hours after administration of the pharmaceutical composition. In some embodiments, the at least one lineage-determining factor is administered to a subject within 30 hours after administration of the pharmaceutical composition. In some embodiments, the at least one lineage-determining factor is administered to a subject within 24 hours after administration of the pharmaceutical composition. In some embodiments, the at least one lineage-determining factor is administered to a subject within 18 hours after administration of the pharmaceutical composition. In some embodiments, the at least one lineage-determining factor is administered to a subject within 12 hours after administration of the pharmaceutical composition. In some embodiments, the at least one lineage-determining factor is administered to a subject within 6 hours after administration of the pharmaceutical composition. In some embodiments, the at least one lineage-determining factor is administered to a subject within 3 hours after administration of the pharmaceutical composition. In some embodiments, the at least one lineage-determining factor is administered to a subject within 1 hour after administration of the pharmaceutical composition.

In some embodiments, the at least one lineage-determining factor is administered to a subject before administration of the pharmaceutical composition. In some embodiments, the at least one lineage-determining factor is administered to a subject within 10 days before administration of the pharmaceutical composition. In some embodiments, the at least one lineage-determining factor is administered to a subject within 9 days before administration of the pharmaceutical composition. In some embodiments, the at least one lineage-determining factor is administered to a subject within 8 days before administration of the pharmaceutical composition. In some embodiments, the at least one lineage-determining factor is administered to a subject within 7 days before administration of the pharmaceutical composition. In some embodiments, the at least one lineage-determining factor is administered to a subject within 6 days before administration of the pharmaceutical composition. In some embodiments, the at least one lineage-determining factor is administered to a subject within 5 days before administration of the pharmaceutical composition. In some embodiments, the at least one lineage-determining factor is administered to a subject within 4 days before administration of the pharmaceutical composition. In some embodiments, the at least one lineage-determining factor is administered to a subject within 72 hours before administration of the pharmaceutical composition. In some embodiments, the at least one lineage-determining factor is administered to a subject within 60 hours before administration of the pharmaceutical composition. In some embodiments, the at least one lineage-determining factor is administered to a subject within 48 hours before administration of the pharmaceutical composition. In some embodiments, the at least one lineage-determining factor is administered to a subject within 42 hours before administration of the pharmaceutical composition. In some embodiments, the at least one lineage-determining factor is administered to a subject within 36 hours before administration of the pharmaceutical composition. In some embodiments, the at least one lineage-determining factor is administered to a subject within 30 hours before administration of the pharmaceutical composition. In some embodiments, the at least one lineage-determining factor is administered to a subject within 24 hours before administration of the pharmaceutical composition. In some embodiments, the at least one lineage-determining factor is administered to a subject within 18 hours before administration of the pharmaceutical composition. In some embodiments, the at least one lineage-determining factor is administered to a subject within 12 hours before administration of the pharmaceutical composition. In some embodiments, the at least one lineage-determining factor is administered to a subject within 6 hours before administration of the pharmaceutical composition. In some embodiments, the at least one lineage-determining factor is administered to a subject within 3 hours before administration of the pharmaceutical composition. In some embodiments, the at least one lineage-determining factor is administered to a subject within 1 hour before administration of the pharmaceutical composition.

In some embodiments, the at least one lineage-determining factor is administered to a subject simultanousely with administration of the pharmaceutical composition.

In some embodiments, the at least one lineage-determining factor is selected from the group consisting of MEF2C, GATA4, TBX5, ASCL1, BRN2, MYTL1, OCT4, KLF4, SOX2, MYC and MYOD. In some embodiments, the at least one lineage-determining factor is MEF2C. In some embodiments, the at least one lineage-determining factor is GATA4. In some embodiments, the at least one lineage-determining factor is TBX5. In some embodiments, the at least one lineage-determining factor is ASCL1. In some embodiments, the at least one lineage-determining factor is BRN2. In some embodiments, the at least one lineage-determining factor is MYTL1. In some embodiments, the at least one lineage-determining factor is OCT4. In some embodiments, the at least one lineage-determining factor is KLF4. In some embodiments, the at least one lineage-determining factor is SOX2. In some embodiments, the at least one lineage-determining factor is MYC. In some embodiments, the at least one lineage-determining factor is MYOD. In some embodiments, the at least one lineage-determining factor comprises or consists ofASCL1, BRN2, and MYTL1. In some embodiments, the at least one lineage-determining factor comprises or consists of MEF2C, GATA4, and TBX5. In some embodiments, the at least one lineage-determining factor comprises or consists of OCT4, KLF4, SOX2, and MYC.

In some embodiments, the damaged tissue is a cardiac tissue and wherein said at least one lineage-determining factor is MEF2C, GATA4, or TBX5. In some embodiments, the damaged tissue is a neural tissue and wherein said at least one lineage-determining factor is ASCL1, BRN2, or MYTL1. In some embodiments, the damaged tissue is a skeletal muscle tissue and wherein said at least one lineage-determining factor is MYOD. In some embodiments, the damaged tissue is a stem cell lineage and wherein said at least one lineage-determining factor is OCT4, KLF4, SOX2, or MYC.

### Cell Transplantation

In another aspect, the present disclosure provides methods for performing cell transplantation in a recipient in need thereof, comprising (i) generating a second cell from a first cell of a donor, wherein the donor is immunocompatible with the recipient, and wherein the second cell is generated according to the methods disclosed herein; and (ii) transplanting the second cell into said recipient.

In some embodiments, the recipient and the donor are the same individual. In some embodiments, the recipient and the donor are different individuals. In some embodiments, the first cell is a fibroblast, an endothelial cell, or a PBMC. In some embodiments, the second cell is a cardiomyocyte-like cell, a cardiomyocyte, a neuron, a skeletal muscle cell, or a stem cell-like cell.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art can alternatively be used.

### EXAMPLES

### Example 1: A Genome-Wide siRNA Screen Identified Barrier Genes

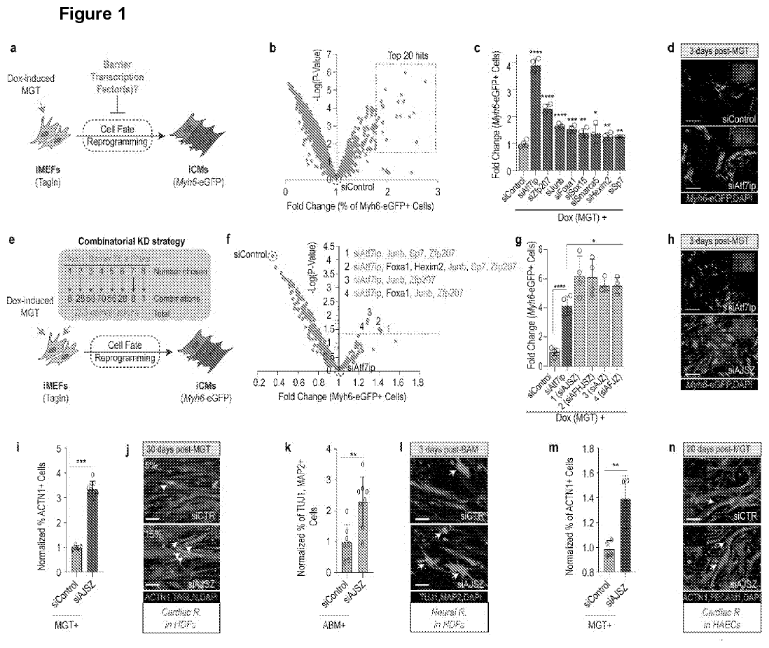

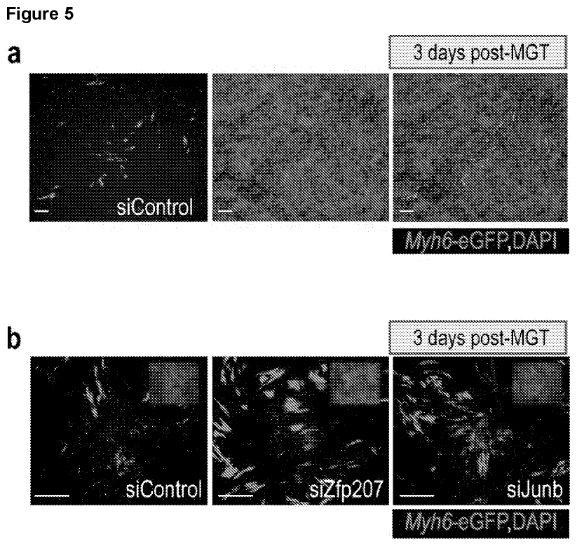

A high-throughput siRNA screen was used to identify TFs that limit cardiac reprogramming (CR) in fibroblasts. As shown in FIG. 1a, an immortalized mouse embryonic fibroblasts (iMGT-Myh6-eGFP-MEFs, referred to hereafter as iMGT-MEFs) that carries a cardiac lineage reporter transgene (Myh6-eGFP) and can be directly reprogrammed into cardiomyocyte-like cells (iCMs) upon doxycycline (Dox)-induced overexpression of the cardiac LDFs Mef2c, Gata4, and Tbx5 (MGT) was utilized. Under conditions optimized for this CR assay, only about ˜6% of fate-challenged cells expressed Myh6-eGFP+ after 3 days of Dox treatment (FIG. 5a), providing an ideal platform for identification of endogenous barriers to CR.

To perform the genome-wide TF screen, iMGT-MEFs were transfected with a library of siRNAs directed against 1435 TFs on day −1, treated with Dox on day 0 to induce MGT expression, and imaged on day 3 for evaluation of CR efficiency (percentage Myh6-eGFP+iCMs) by automated fluorescence microscopy. The screen identified 69 siRNAs that significantly increased CR efficiency (>1.25-fold increase in iCMs, p<0.05) compared with siControl (FIG. 1b and please see submitted supplementary Table 1). The top 20 hits were validated with independent siRNAs, and a final group of siRNAs targeting 8 TFs (Atf7ip, Foxal, Hexim2, Junb, Smarca5, Sox15, Sp7, and Zfp207) were found to robustly increase CR efficiency (1.2-3.8-fold, p<0.05) relative to siControl (FIGS. 1c, 1d and 5b), thus identifying these TFs as barriers to CR in iMEFs.

To determine whether the 8 barrier TFs might act in concert, all possible combinations of the 8 validated siRNAs (255 combinations in total) were tested in the CR assay (FIG. 1e). This analysis identified 4 siRNA combinations that significantly enhanced CR efficiency compared with the single most efficient siRNA, siAtf7ip (FIG. if and please see submitted supplementary Table 2). The combination, consisting of siRNAs against Atf7ip, Junb, Sp7, and Zfp207 increased the abundance of Myh6-eGFP-expressing cells in the CR assay from ˜6% to ˜35-40%, representing an increase in CR efficiency of ˜6-fold compared with siControl and ˜1.5-fold compared with siAtf7ip alone (FIGS. 1g and 1h). These results suggest that Atf7ip, Junb, Sp7, and Zfp207, which are members of the ATF-interacting, AP-1, Sp, and Zinc Finger protein families, respectively, functionally interact to oppose cell fate reprogramming in iMEFs.

### Example 2: AJSZ are Conserved Barriers of Cardiac Reprogramming in Human Fibroblasts

This Example demonstrates that the four genes Atf7ip, Junb, Sp7, and Zfp207 are conserved barrier genes. CR of primary human dermal fibroblasts (HDFs) with MGT was examined to determine whether the barrier function of AJSZ is evolutionarily conserved.

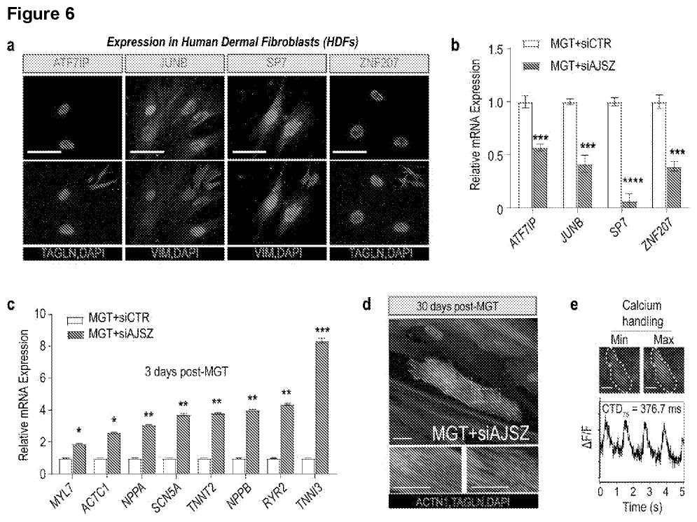

The results confirmed that HDFs express all 4 TFs (FIG. 6a) and that siAJSZ transfection can effectively suppress AJSZ transcription (FIG. 6b). HDFs were transfected with siRNAs on day −1 and transduced with an MGT retrovirus on day 0. qRT-PCR analysis on day 3 demonstrated significantly increased expression of cardiac markers (2-8-fold, p<0.05), including sarcomeric genes (ACTC, MYL7, TNNT2), ion channels (SCN5A, RYR2), and secreted factors (NPPA, NPPB), in siAJSZ-transfected compared with siControl-transfected HDFs (FIG. 6c). Consistent with these findings, immunostaining of the cardiac marker α-actinin (ACTN1) revealed an ˜3.2-fold increase in ACTN1+ cells (from 5% to 16%) among siAJSZ-transfected compared with siControl-transfected HDFs on day 30 after MGT overexpression (p<0.05; FIGS. 1i and 1j). Importantly, the increase in CR efficiency observed after AJSZ KD was specifically accompanied by acquisition of mature CM structural and functional phenotypes, including sarcomeric-like structures and calcium transients (FIGS. 6d and 6e). These results indicate that the function of AJSZ as barriers to CR is conserved between mouse and human fibroblasts.

### Example 3: AJSZ Act as Barriers to Reprogramming in Lineage- and Cell Type-Independent Manners

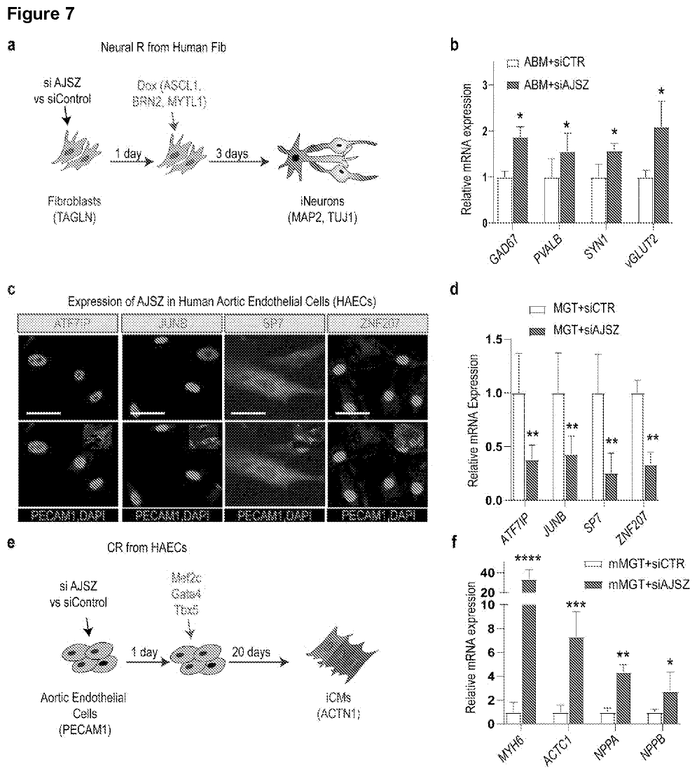

This Example demonstrates that the barrier function of AJSZ was not restricted to cardiac reprogramming but could also increase reprogramming efficiency towards another somatic lineage, such as neural. SiControl- and siAJSZ-transfected HDFs were transduced with a Dox-sensitive lentivirus carrying the neural LDFs Ascl1, Brn2, and Mytl1 (ABM). Compared with siControl-transfected cells, AJSZ KD enhanced by ˜2.3-fold (from 7.3% to ˜17%) the generation of MAP2+ and TUJ1+ neuron-like cells (FIG. 1k, l) and concomitantly increased the transcription of neuron-specific markers21, including vGLUT2, GAD67, PVALB, and SYN1 by ˜2-fold (p<0.05) (FIGS. 7a-7b) on day 3 after ABM overexpression. This indicates that AJSZ-mediated regulation of cell fate stability in human primary fibroblasts is lineage-independent.

Whether the barrier role of AJSZ is conserved in a non-fibroblast cell type, such as endothelial cells was evaluated. First, the inventors confirmed that AJSZ are expressed in primary human aortic endothelial cells (HAECs) (FIG. 7c) and that their expression can be efficiently suppressed by specific siRNAs (FIG. 7d). The inventors found that AJSZ KD in combination with retrovirus-mediated MGT overexpression significantly enhanced cardiac gene expression (2-10-fold, p<0.05) on day 3 (FIGS. 7e-7f) and significantly increased the generation of ACTN1+iCMs by ˜1.5-fold (p<0.05) on day 20 (FIGS. 1m-1n) compared with siControl-transfected HAECs. These results suggest a cell type-independent role for AJSZ as barriers to cell fate reprogramming.

### Example 4: AJSZ-Mediated Barrier Mechanisms are Active at Ground State in Fibroblasts

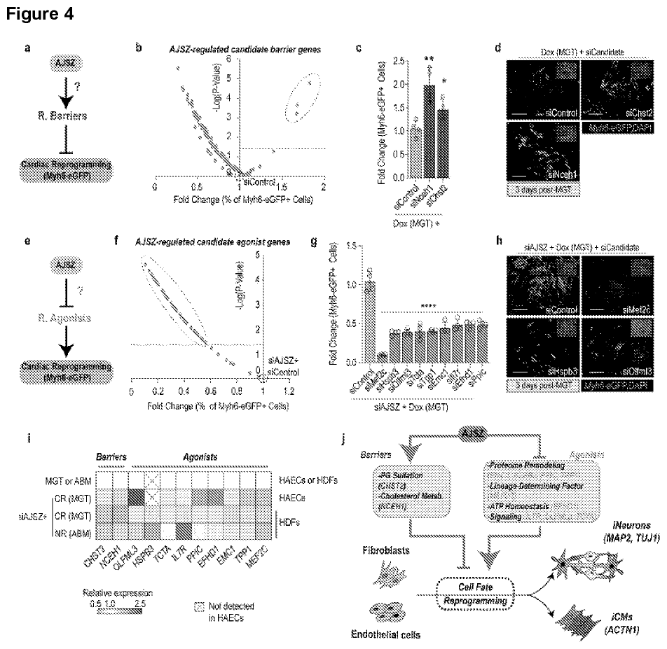

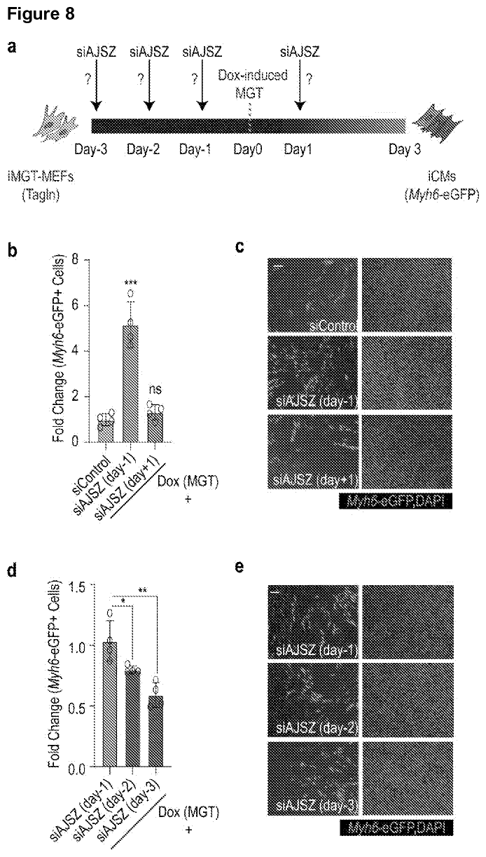

This Example demonstrates that AJSZ-mediated barrier mechanisms are actively operating in fibroblasts at ground state, prior to the TF-induced cell fate challenge. The inventors determined whether the timing of AJSZ KD relative to LDF overexpression would influence reprogramming efficiency (FIG. 8a). While siAJSZ transfection on day −1 relative to MGT induction robustly enhanced CR efficiency, a delay of AJSZ KD to day+1 after MGT overexpression eliminated the improvement in CR efficiency (FIGS. 4b-4c). Moreover, earlier AJSZ KD on day −2 or −3 before MGT overexpression significantly and progressively reduced the ability of AJSZ KD to improve CR efficiency (FIGS. 4d-4e).

### Example 5: AJSZ Pervasively Interact with Chromatin Via Binding to AP-1 Motifs

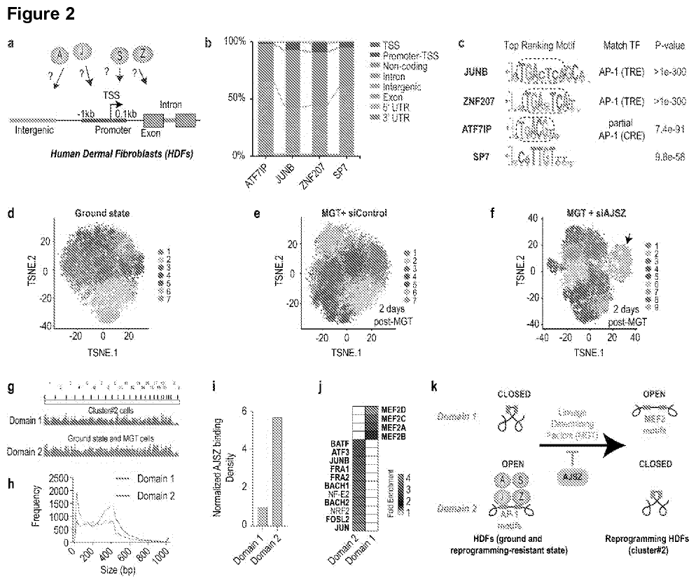

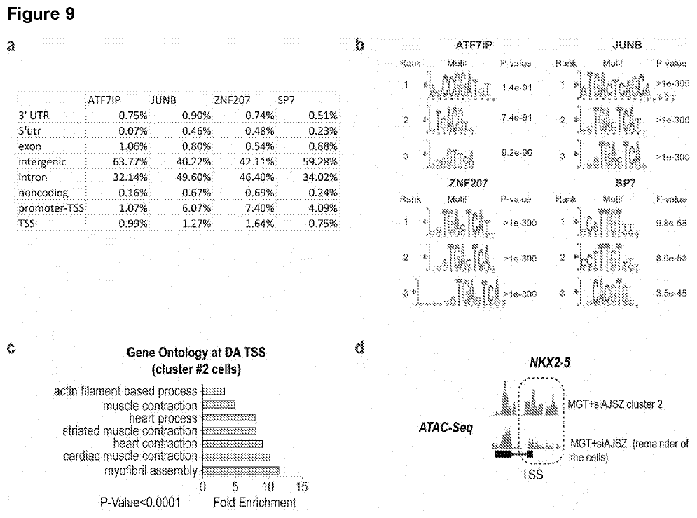

The DNA-binding sites of AJSZ in unchallenged HDFs was characterized using ChIP-seq (FIG. 2a). This analysis identified 91,196 replicated peaks (binding sites) for JUNB, 44,100 for ATF7IP, 19,169 for SP7, and 4135 for ZNF207 across two duplicates. Examination of the genomic distribution of AJSZ-binding sites revealed that most interactions (88-95%) occurred within intronic and intergenic regions (FIG. 2b and FIG. 9a). Without being bound to any particular theory, this suggests that AJSZ-mediated barrier mechanisms might primarily consist of long-range interactions and/or structural roles in the regulation of chromatin organization. Motif enrichment analysis (Homer) revealed that JUNB and ZNF207 preferentially bound to chromatin via prototypical AP-1 (TRE: TGACTCA) motifs, whereas ATF7IP interacted via partial AP-1 motifs (CRE: TGACGT-) (FIG. 2c and FIG. 9b) in HDFs. Thus, binding to AP-1 motifs is a distinctive molecular hallmark of AJSZ interactions with chromatin in HDFs at ground state.

### Example 6: AJSZ Alter Chromatin Remodeling Via Binding to AP-1 Motifs During Cell Fate Reprogramming

This Example demonstrates that, without being bound to any particular theory, the interaction between AJSZ and AP-1 motifs might contribute to oppose chromatin remodeling induced by LDFs during cell fate reprogramming. scATAC-seq was performed to generate single-cell chromatin accessibility profiles from HDFs at ground state (untreated) or 2 days after MGT overexpression in siControl- or siAJSZ-transfected HDFs. t-distributed stochastic neighbor embedding (t-SNE) clustering of HDFs at ground state (20,627 cells) and MGT+siControl cells (15,859 cells), showed that cell clusters were distributed as a compact continuum (FIGS. 2d-2e), indicating that the chromatin accessibility profile among the different cell populations did not differ markedly. In contrast, t-SNE clustering of MGT+siAJSZ cells (8966 cells) identified a discrete cell population (cluster #2) with a chromatin accessibility profile that diverged markedly from the remaining cell populations (black arrow, FIG. 2f). This cluster represented ˜13% of total cells examined under this condition, which is notably similar to the observed percentage of ACTN1+iCMs (˜16%) generated after siAJSZ transfection and MGT overexpression in the CR assay (FIGS. 1i-1j). Comparison of Gene Ontology (GO) analysis of genes with differentially accessible (DA) transcriptional start sites (TSSs) revealed a 5-10-fold enrichment (p<0.0001) of cardiac terms in cluster #2 compared with clusters 1 and 3-7 (FIG. 9c and please see submitted supplementary Table 3). These terms included striated muscle contraction and myofibril assembly and involved a wide array of cardiac-specific genes such as NKX2-5, ACTA1, and NPPA (FIG. 9d). Collectively these results indicate that 1) cluster #2 represent a cell population displaying epigenetic hallmarks indicative of CR and 2) that AJSZ regulate chromatin accessibility dynamics during this process.

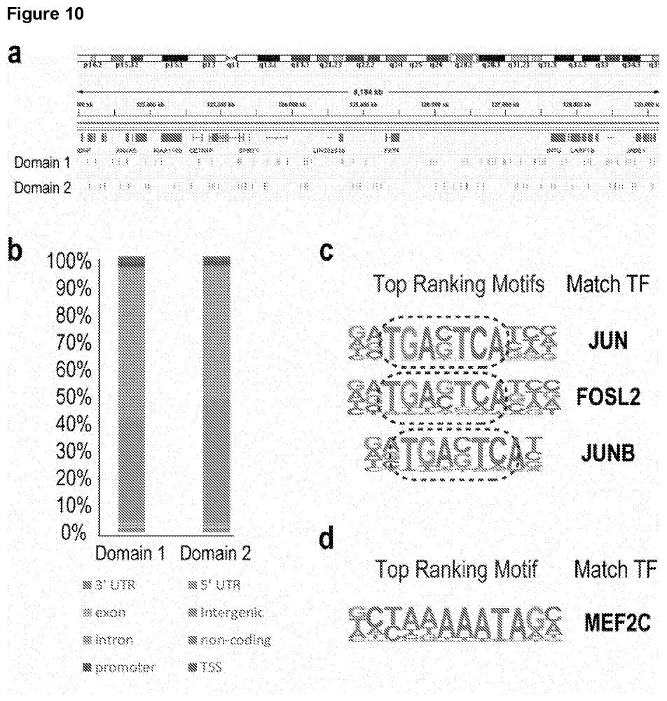

All regions of DA chromatin in cells with reduced AJSZ levels undergoing reprogramming (cluster #2 cells from siAJSZ+MGT HDFs; domain 1) and cells with normal AJSZ levels in a non-reprogrammed or reprogramming-resistant state (unchallenged HDFs or siControl+MGT HDFs; domain 2) were mapped. Interestingly, the two mutually exclusive domains were evenly scattered across the genome (FIG. 2g and FIGS. 10a-10b) and were composed of short regions (<600 bp) of DA chromatin (FIG. 2h), spanning ˜4.5 Mbp and ˜7 Mbp for domain 1 and domain 2, respectively. These findings indicated that, without being bound by any specific theory, AJSZ regulate chromatin accessibility on a genome-wide scale during cell fate reprogramming. Next, AJSZ binding frequency to either domains in HDFs at ground state was compared to determine whether AJSZ could play a direct role in the regulation of domain 1 or 2 chromatin accessibility. Integration of scATAC-seq and ChIP-seq data revealed a markedly higher frequency of AJSZ binding to domain 2 compared with domain 1 chromatin (5.7-fold, FIG. 2i). Consistent with this observation, motif enrichment analysis in domain 2 showed ˜4-fold enrichment of AP-1 motifs (1 motif/kb) in domain 2 compared with domain 1 (FIG. 2j), and notably, these AP-1 motifs contained the same sequence (TGACTCA) (FIG. 10c and please see supplementary Table 4) that was identified for JUNB and ZNF207 chromatin binding by ChIP-Seq (FIG. 2c). Conversely, motif enrichment analysis in domain 1 revealed ˜3.5-fold enrichment of MEF2 motifs in domain 1 (0.6 motif/kb) compared with domain 2 (FIG. 2j) and included the canonical binding sequence for the cardiac LDF MEF2C (FIG. 10d and please see supplementary Table 4). Without being limited to any specific theory, these results are consistent with a model in which genome-wide binding of AJSZ to AP-1 motifs in domain 2 chromatin impairs the ability of the LDF MEF2C to remodel chromatin and promote domain 1 opening, thereby opposing cell fate conversion (FIG. 2k).

### Example 7: AJSZ Play a Proximal Role in Regulating Transcription During Cell Fate Reprogramming

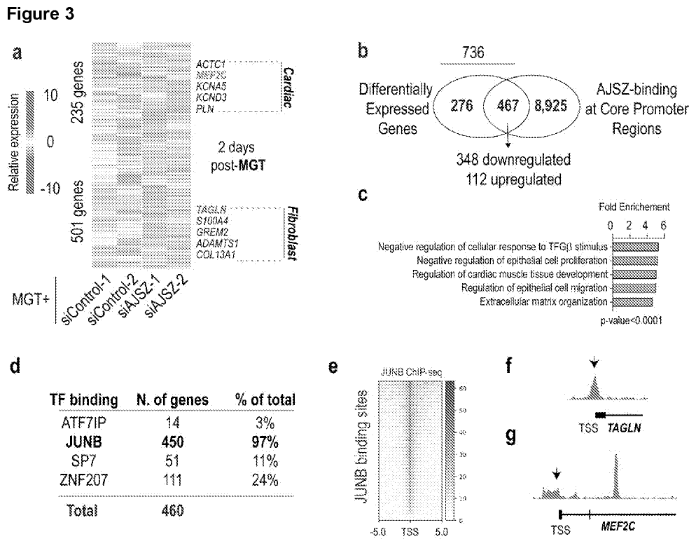

Although AJSZ binding to promoter TSS regions (−1 kbp, +0.1 kbp) accounted for <10% of total AJSZ-chromatin interactions in HDFs at ground state (FIG. 2e and FIG. 10a), a total of 9392 core promoter regions were bound by AJSZ (please see supplementary Table 5). Genome-wide RNA-seq of control and AJSZ KD HDFs 2 days after MGT overexpression was performed. 736 differentially expressed (DE) genes were identified, of which 501 and 235 were downregulated and upregulated, respectively by AJSZ KD (p<0.05, FIG. 3a and please see submitted supplementary Table 6). After integration of the RNA-seq and ChIP-seq datasets, ˜2/3 of the DE genes (460 of 736) were found bound at their core promoter regions by at least one of the 4 barrier TFs in HDFs (FIG. 3b and please see submitted supplementary Table 7). Notably, core promoter binding correlated with gene downregulation for ˜75% of the DE genes (FIG. 3b), thus indicating a predominantly activating role for AJSZ in the regulation of transcription during cell fate reprogramming. In this context, GO analysis of core promoter-bound DE genes revealed a significant enrichment of terms related to cell fate specification, cardiac muscle differentiation, fibroblast proliferation, collagen organization and TGFβ signaling, substantiating the potential involvement of AJSZ in the control of cell fate-regulating transcriptional programs in fibroblasts (FIG. 3c and please see submitted supplementary Table 8). Moreover, assessment of individual AJSZ TF contributions to core promoter binding revealed that 97% of the promoters were bound by JUNB in HDFs (FIG. 3d). In this context, analysis of binding site distribution showed that JUNB binding was centered at the TSS of targeted promoters (FIG. 3e) and could be observed for both downregulated (TAGLN) and upregulated (MEF2C) genes (FIGS. 3f-3g). Collectively, these results show that, without being bound to any specific theory, AJSZ play a proximal role, via JUNB binding to the TSS of DE genes, in the regulation of fibroblast (TGFβ, collagen organization, proliferation) and cell fate-modulating transcriptional programs during reprogramming.

### Example 8: AJSZ Oppose Cell Fate Reprogramming Via Transcriptional Regulation of a Network of Agonists and Barriers

Given that AJSZ act as both barriers and proximal regulators of transcription during cell fate reprogramming (FIGS. 1 and 3), they might promote cell fate stability by upregulating reprogramming barrier genes (FIG. 4a), while downregulating genes required for reprogramming (=agonists). Top 25 percentile core promoter-bound and downregulated genes were tested (please see submitted supplementary Table 9), in MGT+siAJSZ condition as compared to MGT+siControl, for barrier to reprogramming function using a siRNA-mediated KD strategy in the iMGT-MEF CR assay. Two hits were identified, siChst2 and siNceh1, that robustly enhanced CR efficiency (1.5- and 2-fold, p<0.05 and p<0.01, respectively; FIGS. 4b-4d), thus uncovering a novel barrier to reprogramming roles for carbohydrate sulfotransferase 2 (Chst2) which mediates 6-0 sulfation within proteoglycan structures, and neutral cholesterol ester hydrolase 1 (Ncehl) which regulates lipid droplet formation and platelet-activating factor synthesis. siRNAs against top 25 percentile core promoter-bound and upregulated genes were screened to identify AJSZ-regulated reprogramming agonists (FIG. 4e), in MGT+siAJSZ condition as compared to MGT+siControl (please see submitted supplementary Table 10), for reprogramming requirement in the siAJSZ-induced CR assay. This approach identified 61 siRNAs that reduced CR efficiency induced by AJSZ KD by >50% (FIG. 4f); of these, 9 siRNA hits had no effect on cell viability and were selected for further analysis (FIGS. 4g-4h). The 9 agonists could be grouped into 4 gene categories involved in regulation of cell fate specification (Mef2c), protein folding and degradation (Emc1, Hspb3, Ppic, Tpp1), inflammation and TGFβ signaling (I17r, Olfml3, Tcta), and ATP homeostasis (Efhd1) (FIGS. 4g-4h). These data indicate that, without being bound by any particular theory, AJSZ oppose cell fate reprogramming by coordinating the expression of a downstream network of reprogramming agonists and barriers.

### Example 9: AJSZ-Mediated Regulation of the Barrier Network is Lineage- and Cell Type-Independent

This Example demonstrates that the AJSZ-mediated regulation of the barrier network was not lineage and/or cell type-specific. The expression of the 9 agonists and 2 barriers in neural and cardiac reprogramming assays with HDFs and HAECs after AJSZ KD was evaluated. Remarkably, AJSZ KD led to a lineage- and cell type-independent downregulation of the barrier genes (CHST2 and NCEH1), coincident with conserved upregulation of reprogramming agonists (MEF2C, TPP1, EMC1, PPIC, IL7R, and EFHD1) after 3 days of reprogramming (FIG. 4i). These results demonstrate that, without being limited to any specific theory, AJSZ proximally orchestrate the expression of a conserved barrier network specialized in the regulation of cellular processes (e.g., cell fate specification, proteome remodeling, proteoglycan sulfation) controlling cell fate stability in differentiated cells (FIG. 4j).

The above Examples demonstrates that 4 TFs (AJSZ) were identified that oppose cell fate reprogramming in a lineage- and cell type-independent manner. Materials and methods

iMGT-Myh6-eGFP-MEFs and Screening Assays

Immortalized Dox-inducible iMGT-Myh6-eGFP-MEFs were described previously. The cells were cultured in plates precoated with 0.1% gelatin (Stem Cell Technologies, 7903) and maintained in Fibroblast Culture Medium (FCM) consisting of DMEM (Coming, 10-013-CV), 10% fetal bovine serum (FBS; VWR, 89510-186), and 1% penicillin/streptomycin solution ((10,000 U/mL), Catalog #: 15140122) at 37° C. in a 5% CO2 atmosphere. One day prior to siRNA transfection (day −1), cells were detached by addition of 0.25% trypsin-EDTA (Thermo Fisher, 25200056) for 3 min at 37° C., and then washed in FCM, centrifuged, and resuspended in Induced-CM Reprogramming Medium (iCRM), consisting of DMEM, 20% Medium 199 (Gibco, 11150-059), 10% FBS, and 1% P/S. Cells were plated in 384-well plates at 103 cells/well and transfected with an siRNA library directed against 1435 mo use TFs (Dharmacon-Horizon Discovery; siGenome-siRNA library, G-015800). The next day (day 0), 1 μg/mL doxycycline hydrochloride (Dox; Sigma, D3072) diluted in iCRM was added to the cells to induce MGT expression. On day 3, the cells were fixed with 4% paraformaldehyde (PFA) and processed for immunostaining, microscopy, and Myh6-eGFP quantification. The top 20 siRNA hits were validated using independent siRNAs (Dharmacon-Horizon Discovery; ON-Target plus pooled siRNAs). All possible combinations of the top 8 siRNAs (255 combinations) were assembled by echo-spotting using an Echo 550 liquid handler (Labcyte) and the cells were processed as described above. All experiments were performed in quadruplicate. For follow-up analyses, the cells were collected on 3 after Dox addition for qRT-PCR analysis.

### Human Primary Dermal Fibroblasts (HDFs)

Newborn human primary foreskin fibroblasts were obtained from American Type Culture Collection (ATCC; CRL-2097, CCD-1079Sk) and cultured in plates precoated with 0.1% gelatin in FCM. For cardiac reprogramming, the cells were allowed to reach 80% confluency, harvested using trypsin-EDTA as described above, resuspended in iCRM, added to 384-well plates at 103 cells/well, and transfected with the indicated siRNAs. The next day (day 0), the cells were transduced by addition of 1 μL/well of mouse MGT retrovirus17 diluted in iCRM. Cells were collected on day 2 for RNA-seq and scATAC-seq experiments, on day 3 days for qRT-PCR experiments, and on day 30 for calcium handling assays. Immunostaining was performed on the days indicated in the legends. During the incubations, 50% of the iCRM medium was exchanged every other day up to day 8, and was then replaced with RPMI 1640 (Life Technologies, 11875093), 1% B27 supplement (Life Technologies, 17504044), and 1% P/S. Neuronal reprogramming was induced as previously described(Vierbuchen et al., 2010; Mahmoudi et al., 2019). In brief, HDFs were cultured and harvested as described above, resuspended in FCM, added to 384-well plates at 3×103 cells/well, and transfected with siRNAs. The next day (day −1), 0.25 μL of F-ABM lentiviral mix (1:1:1:1 of Addgene plasmids #27150, Tet-O—FUW-Ascl1; #27151, Tet-O-FUW-Brn2; #27152, Tet-O-FUW-Mytl1; #20342, FUW-M2rtTA) was added to each well, and the cells were incubated for an additional 24 h. The following day (day 0), ABM expression was induced by addition of 2 μg/mL Dox diluted in FCM. On day 2, media was replaced by Minimal N euronal Medium consisting of DMEM/F-12 (Gibco, 11220-032), 1% B27, 1% N2 Supplement (Gibco, 17502-048), and 1% human recombinant insulin and zinc solution (Gibco, 12585-014). On day 3, cells were harvested and processed for immunostaining or qRT-PCR analysis.

### Human Adult Aortic Endothelial Primary Cells (HAECs)

Human adult aortic endothelial cells (HAECs) were obtained from ATCC (PCS-100-011) and cultured in plates precoated with 0.1% gelatin in Vascular Cell Basal Medium (VCBM; ATCC, PCS-100-030) containing endothelial cell growth kit-BBE (ATCC, PCS-100-040) and 0.1% P/S. When the cells reached 80% confluency, they were harvested with trypsin-EDTA solution for primary cells (ATCC, PCS-999-003) for 3 min at 37° C., washed with trypsin-neutralizing solution (ATCC, PCS-999-004), resuspended in VCBM/kit-BBE medium, added to 384-well plates at 3×103 cells/well, and transfected with siRNAs. One day later (day 0), cells were transduced by addition of 1 μL/well of mouse MGT retrovirus. The cells were collected on day 3 for qRT-PCR analysis and on day 20 for immunostaining. During the incubation, 50% of VCBM/kit-BBE medium was exchanged every other day starting on day 4.

siRNA Transfection

Mouse and human siRNAs were purchased from Dharmacon or Ambion and added at a final concentration of 25 nM. A negative control siRNA (referred to as siCTR or siControl) was obtained from Dharmacon. Transfection was performed using Opti-MEM (Gibco, 31985070) and Lipofectamine RNAiMAX (Gibco, 13778150) according to the manufacturer's instructions. siRNA transfection was performed on day −1, unless otherwise noted.

### Immunostaining and Fluorescence Microscopy

Cells were fixed with 4% PFA in phosphate-buffered saline (PBS) for 30 min and blocked with blocking buffer consisting of 10% horse serum (Life Technologies, 26050088), 0.1% gelatin, and 0.5% Triton X-100 (Fisher Scientific, MP04807426) in PBS for 30 min. Cells were incubated with primary antibodies overnight at 4° C., and then with secondary antibodies plus 4′,6-diamidino-2-phenylindole (DAPI; Sigma, D9542) for 1 h in the dark at room temperature. Cells were washed with PBS between each step. Cell were imaged with an Image Xpress confocal microscope (Molecular Devices) and fluorescence was quantified with Molecular Device software. Experiments were performed in quadruplicate.

The primary antibodies used were: rabbit anti-ATF7IP (Sigma, HPA023505, 1:200); rabbit anti-ATF7IP (Invitrogen, PA5-54811, 1:200); rabbit anti-JUNB (Abcam, Ab128878, 1:200); rabbit anti-SP7/OSTERIX (Abcam, Ab22552, 1:500); mouse anti-ZNF207 (Sigma, SAB1412396, 1:500); rabbit anti-TAGLN (Abcam, Ab14106, 1:800); mouse anti-VIMENTIN (Santa Cruz Biotechnology, sc-373717, 1:800); goat polyclonal anti-TAGLN (GeneTex, GTX89789, 1:800); mouse anti-ACTN1 (Sigma, A7811, 1:800); goat anti-PECAM1 (H3) (Santa Cruz Biotechnology, Sc1506, 1:200); rabbit anti-MAP2 (Abcam, Ab32454, 1:200); and mouse anti-TUJ1 (R&D Systems, MAB1195, 1:200). Secondary antibodies were: Alexa Fluor 488 goat-anti-rabbit IgG (H+L) (Invitrogen, A1008, 1:1000); Alexa Fluor 488 donkey-anti-mouse IgG (H+L) (Invitrogen, A21202 1:1000); Alexa Fluor 568 goat-anti-mouse IgG (H+L) (Invitrogen, A10037, 1:1000); Alexa Fluor 568 donkey anti-goat IgG (H+L) (Invitrogen, A11057, 1:1000); Alexa Fluor 680 donkey-anti-mouse IgG (H+L) (Invitrogen, A10038, 1:1000); and Alexa Fluor 680 donkey-anti-rabbit IgG (H+L) (Invitrogen, A10043, 1:1000).

RNA Extraction and qRT-PCR

Total RNA was extracted using TRIzol reagent (Invitrogen, 15596026) and chloroform (Fisher Chemical, C298-500) following the manufacturers' recommendations. RNA in the aqueous phase was precipitated with isopropanol, centrifuged, washed with 70% ethanol, and eluted in DNase- and RNase-free water. RNA concentration was measured by Nanodrop (Thermo Scientific). Aliquots of 1 μg of RNA were reverse transcribed using a QuantiTect Reverse Transcription kit (Qiagen, 205314), and qPCR was performed with iTaq SYBR Green (Life Technologies) using a 7900HT Fast Real-Time PCR system (Applied Biosystem). Gene expression was normalized to that of glyceraldehyde 3-phosphate dehydrogenase (GAPDH) for human samples or R-actin (Actb) for mouse samples using the 2-AACt method. Human and mouse primer sequences for qRT-PCR were obtained from Harvard Primer Bank. Primers were: ATF 71P (#38261961c1), JUNB (#44921611c1), SP7 (#22902135c2), ZNF207 (#148612834c1), ACTC1 (#113722123c1), MYL7 (#50593014c1), NPPA (#23510319al), NPPB (#83700236c1), RYR2 (#112799846c1), SCN5A (#237512981c1), TNNI3 (#151101269c1), TNNT2 (#48255880c1), GAD67 (#58331245c2), PVALB (#55925656c2), SYN1 (#91984783c1), vGLUT2 (#215820654c2), MYH6 (#289803014c3), HSPB3 (#306966173c2), OLFML3 (#50593011c1), TCTA (#148922970c1), TPP1 (#118582287c1), EMC1 (#22095330c3), IL7R(#28610150c2), EFHD1 (#237649043bl), PPIC (#45439319c2), NCEH1 (#226423949c2), CHST2 (#344925865c1), and GAPDH (#378404907c1).

### Retrovirus and Lentivirus Preparation

Large-scale retrovirus production was performed at the SBP Viral Vector Core Facility SBP as previously described. Briefly, for retrovirus preparation, Retrovector, Retro-Gag-Pol, and pMD2.G plasmids were co-transfected into HEK-293T cells at a ratio of 3:2:1. For lentivirus preparation, shZfp207 lentivector plasmid DNA was co-transected with pCMVDR8.74 and pMD2.G into HEK-293T cells using the calcium phosphate method. UltraCulture serum-free medium (Lonza) supplemented with 1 mM L-glutamine (Life Technologies) was used to re-feed transfected cells, and the supernatant was collected every 24 h from day 2 to day 4 after transfection. Viral supernatants were pooled, passed through a 0.22-μm-pore filter, concentrated, and purified by 20% sucrose gradient ultracentrifugation at 21,000 rpm for 2 h at 4° C. The pellet containing concentrated viral particles was resuspended in PBS, aliquoted, and kept at −80° C.

### Calcium Handling Assay

The calcium assay was performed on day 30 (HDFs) or day 20 (HAECs) after MGT overexpression. The assay was performed using the labeling protocol as previously described46. Briefly, 50% of the cell culture supernatant was replaced with a 2× calcium dye solution consisting of Fluo-4 NW dye (Invitrogen, F36206), 1.25 mM probenecid F-127 (Invitrogen), and 100 μg/mL Hoescht 33258 (Invitrogen, H3569, in water) diluted in warm Tyrode's solution (Sigma), and the cells were incubated at 37° C. for 45 min. The cells were then washed 4 times with fresh pre-warmed Tyrode's solution and automatically imaged with an ImageXpress Micro XLS microscope (Molecular Devices) at an acquisition frequency of 100 Hz for a duration of 5 s with excitation 485/20 nm and emission 525/30 nm filters. A single image of Hoescht fluorescence was acquired before the time series. Fluorescence quantification over time and single-cell trace analysis were automatically performed using custom software packages developed by Molecular Devices and the Colas laboratory.

### RNA-Seq and Data Analysis

HDFs were added to 384-well plates at 103 cells/well and transfected with siRNAs (siATF7IP, siJUNB, siSP7, siZNF207 individually or in combination) in iCRM. The next day (day 0), the cells were transduced with 1 μL/well mouse MGT retrovirus diluted in iCRM. On day 2, cells were collected and RNA was extracted using TRIzol. Cells were pooled from 16 wells per sample, and two biological replicates per condition were analyzed. Library preparation was performed by Novogene using their in-house preparation protocol. Briefly, mRNA was enriched using oligo (dT) beads and fragmented randomly using a fragmentation buffer. cDNA was generated from mRNA template using a random hexamer primer followed by second-strand synthesis. Terminal repair, A ligation, and sequencing adaptor ligation were then performed. The final libraries were generated through size selection and PCR enrichment and sequenced as 2×150 bp on a HiSeq2500 Sequencer (Illumina). Samples were sequenced to an approximate depth of 35-40 million reads per sample. Raw sequencing reads were trimmed using Trimmomatic (0.36) with a minimum quality threshold of 35 and minimum length of 36. Processed reads were mapped to the hg38 reference genome using HISAT2 (2.0.4). Counts were then assembled using Subread featureCounts (1.5.2). Differential gene expression was analyzed using the DESeq2 package (1.20) in R. Genes were defined as differentially expressed if the adjusted p value was <0.05 after correction for multiple testing using the Benjamini-Hochberg method. Gene Ontology (GO) analysis was performed using PANTHER version 12.0 classification.

Single-Cell ATAC-Seq (scATAC-Seq)

scATAC-seq experiments were performed with control (untreated) HDFs, MGT+siControl HDFs, and MGT+siAJSZ HDFs. HDFs were added to 384-well plates at 2.5×103 cells/well in iCRM and transfected with siRNAs. The next day (day 0), cells were transduced mouse MGT retrovirus and collected 2 days later using trypsin-EDTA. Cells from 40 wells were pooled to obtain ≥2×105 cells per sample, with two biological replicates per condition. Cells were washed with FCM, centrifuged in conical tubes, and the pellets were frozen in Freezing Medium (DMEM, 10% DMSO, 20% FBS), transferred to cryotubes, and placed in Mr, Frosty containers (Thermo Fisher) at −80° C.

Samples were processed for scATAC-seq at UCSD CMME. Samples were processed for scATAC-seq at UCSD CMME using 10× Genomics and sequenced on a NovaSeq 6000 at a depth of 20-25k read pairs per nucleus. CellRanger-ATAC (1.1.0) pipeline was used to filter and align reads, count barcodes, identify transposase cut sites, detect chromatin peaks, prepare t-SNE dimensionality reduction plots, and compare differential accessibility between clusters. The CellRanger-ATAC pipline uses the following tools: cutadapt, BWA-MEM, SAMtools tabix, and bedtools. Further differential accessibility analysis was performed using DiffBind (2.12.1) and custom R scripts and visualized with ggplot2. Tracks were visualized using Integrative Genome Viewer 2.8.12. All scripts for this analysis are available on GitHub [https://github.com/smurph50].

### Chromatin Immunoprecipitation-Seq (ChIP-Seq)

ChIP-seq experiments were performed with HDFs using a SimpleChip Plus sonication chromatin IP kit (Cell Signaling Technology, 56383) according to the manufacturer directions. In brief, HDFs were grown to 80% confluency (107/sample) and then crosslinked with 1% formaldehyde (Sigma, F8775) in PBS at room temperature for 20 min with occasionally stirring. The crosslinking reaction was quenched by addition of 0.125 M glycine for 10 min, and chromatin was fragmented for 25 min using a Bioruptor Pico sonicator (Diagenode) to an average DNA fragment length of 200-500 bp. DNA was quantified with Qubit (Invitrogen, Q32854). Samples equivalent to 100 μg of DNA were incubated overnight at 4° C. with 4 μg of rabbit polyclonal anti-ATF7IP (Invitrogen, PA5-54811), rabbit monoclonal anti-JUNB (C37F9) (Cell Signaling Technology, 3753S), rabbit anti-SP7/OSTERIX (Abcam, Ab22552), or rabbit polyclonal anti-ZNF207 (Bethyl laboratories, A305-814AM). Normal rabbit IgG (Cell Signaling Technology, 2729) was used as a negative control. Immunocomplexes were captured by rotation with protein G-coupled magnetic beads (Cell Signaling Technologies, 9006) for 2 h at 4° C., and the immunoprecipitated genomic DNA was collected by incubation with 50 μL elution buffer. Library preparation and sequencing of immunoprecipitated and input DNA was performed by UCSD IGM core facility. Raw reads were mapped to GRCh38 using Bowtie2 (2.3.5). Since each sample was run across two lanes, SAM files were merged using Picard (2.20.5). MACS2 (2.1.1) was used to call narrow peaks relative to input with a q value cutoff of 0.01. Peaks were annotated with Homer (4.10.4) and motifs were analyzed using MEME-ChIP (5.1.1). BigWig files were generated using Deeptools (2.2) bamCoverage. Tracks were visualized with Fluff (biofluff 3.0.3). Gene ontology biological process terms were found with PANTHER GO and overlap analyses were performed using custom R scripts with venneuler and ggplot2 packages. Bedtools (v2.29.2) was used for genomic comparisons and combining ChIP-seq and scATAC-seq data. HOMER was used to find motifs with a scrambled background.

### Statistical Analysis

All statistical analyses were performed using Prism version 8.0 (GraphPad Software, San Diego CA, USA). Data are presented as the mean±SEM unless noted. Statistical significance was analyzed by unpaired Student's t-test or one-way ANOVA. P values of <0.05 were considered significant.

What is claimed is:

1. A method for generating a cell of interest from a cell of a different cell type, comprising reducing expression level(s) of at least one barrier gene selected from the group consisting of ATF7IP, JUNB, ZNF207, Sp7, FOXA1, HEXIM2, SMARCA5, SOX15, CHST2 and NCEH1 in said cell of a different cell type compared to a wild-type cell of said cell of a different cell type; wherein when said at least one barrier gene is ATF7IP or SOX15, at least a second barrier gene is included.

2. A method for generating a cell of interest from a cell of a different cell type, comprising reducing expression level(s) of at least two barrier gene selected from the group consisting of ATF7IP, JUNB, ZNF207, and Sp7 in said cell of a different cell type compared to a wild-type cell of said cell of a different cell type.

3. The method of claim 1 or 2, wherein said at least one barrier gene comprises ATF7IP, JUNB, and ZNF207.

4. The method of claim 3, wherein said at least one barrier gene further comprises Sp7.

5. The method of any one of claims 1-4, further comprising increasing an expression level of at least one reprogramming agonist gene in said cell of a different cell type compared to a wild-type cell of said cell of a different cell type.

6. The method of claim 5, wherein said at least one reprogramming agonist gene is selected from the group consisting of MEF2C, HSPB3, TPP1, EMC1, PPIC, IL7R, OLFML3, TCTA, and EFHD1.

7. The method of any one of claims 1-6, further comprising increasing an expression level of at least one lineage-determining factor.

8. The method of claim 7, wherein said expression level of said at least one lineage-determining factor is increased within 72 hours after said expression level(s) of said at least one barrier gene is reduced.

9. The method of claim 8, wherein said expression level of said at least one lineage-determining factor is increased within 48 hours after said expression level(s) of said at least one barrier gene is reduced.

10. The method of claim 9, wherein said expression level of said at least one lineage-determining factor is increased within 24 hours after said expression level(s) of said at least one barrier gene is reduced.

11. The method of any one of claims 1-10, wherein said cell of a different cell type is a fibroblast, an endothelial cell, or a PBMC.

12. The method of any one of claims 1-11, further comprising altering chromatin structure of said cell of a different cell type compared to a wild-type cell of said cell of a different cell type.

13. The method of claim 12, wherein said cell of a different cell type has a reduced number of motifs bound to a protein expressed by said at least one barrier gene compared to a wild-type cell of said cell of a different cell type.

14. The method of claim 13, wherein said motifs comprise an AP-1 motif.

15. The method of any one of claims 1-14, wherein said cell of interest is a cardiomyocyte-like cell, a cardiomyocyte, a neuron, a skeletal muscle cell, or an induced pluripotent stem cell.

16. The method of any one of claims 7-15, wherein said at least one lineage-determining factor is selected from the group consisting of MEF2C, GATA4, TBX5, ASCL1, BRN2, MYTL1, OCT4, KLF4, SOX2, MYC, and MYOD.

17. The method of any one of claims 1-16, wherein said cell of interest has an increased expression of at least one cardiac marker as compared to said cell of a different cell type.

18. The method of claim 17, wherein said at least one cardiac marker is selected from the group consisting of ACTC1, MYL7, TNNT2, SCN5A, RYR2, NPPA, and NPPB.

19. The method of any one of claims 1-18, wherein said cell of interest has an increased expression of at least one stem cell marker as compared to said cell of a different cell type.

20. The method of claim 19, wherein said at least one stem cell marker is selected from the group consisting of SSEA4 and NANOG.

21. The method of any one of claims 1-20, wherein said cell of interest has an increased expression of at least one neuron-specific marker as compared to said cell of a different cell type.

22. The method of claim 21, wherein said at least one neuron-specific marker is selected from the group consisting of vGLUT2, GAD67, PVALB, and SYN1.

23. The method of any one of claims 1-22, wherein said reducing expression level(s) of at least one barrier gene comprises contacting said cell of a different cell type with at least one siRNA molecule targeting said at least one barrier gene.

24. The method of any one of claims 1-23, wherein said cell of a different cell type is a somatic cell.

25. The method of any one of claims 1-23, wherein said cell of interest is a somatic cell.

26. The method of any one of claims 1-23, further comprising converting said cell of a different cell type into an induced pluripotent stem cell (iPSC) state.

27. A pharmaceutical composition, comprising at least one inhibitor of at least one barrier gene selected from the group consisting of ATF7IP, JUNB, ZNF207, Sp7, FOXA1, HEXIM2, SMARCA5, SOX15, CHST2 and NCEH1, and a pharmaceutically acceptable carrier or excipient; wherein when said at least one barrier gene is ATF7TP or SOX15, at least a second inhibitor of a second barrier gene is included.

28. A pharmaceutical composition, comprising at least one inhibitor of at least two barrier gene selected from the group consisting of ATF7IP, JUNB, ZNF207, and Sp7, and a pharmaceutically acceptable carrier or excipient.

29. The pharmaceutical composition of claim 27 or 28, wherein said at least one barrier gene comprises ATF7TP, JUNB, and ZNF207.

30. The pharmaceutical composition of claim 29, wherein said at least one barrier gene further comprises Sp7.

31. The pharmaceutical composition of any one of claims 27-30, wherein said at least one inhibitor comprises at least one siRNA molecule targeting said at least one barrier gene.

32. A method for repairing or restoring functional and structural integrity to a damaged tissue in a subject in need thereof, comprising administering to said subject an effective amount of the pharmaceutical composition of any one of claims 27-31.

33. The method of claim 32, further comprising administering at least one lineage-determining factor.

34. The method of claim 33, wherein said at least one lineage-determining factor is administered within 72 hours after said pharmaceutical composition is administered.

35. The method of claim 34, wherein said at least one lineage-determining factor is administered within 48 hours after said pharmaceutical composition is administered.

36. The method of claim 35, wherein said at least one lineage-determining factor is administered within 24 hours after said pharmaceutical composition is administered.

37. The method of any one of claims 32-36, wherein said damaged tissue is a cardiac tissue and wherein said at least one lineage-determining factor is MEF2C, GATA4, or TBX5.

38. The method of any one of claims 32-36, wherein said damaged tissue is a neural tissue and wherein said at least one lineage-determining factor is ASCL1, BRN2, or MYTL1.

39. The method of any one of claims 32-36, wherein said damaged tissue is a skeletal muscle tissue and wherein said at least one lineage-determining factor is MYOD.

40. The method of any one of claims 32-36, wherein said damaged tissue is a stem cell lineage and wherein said at least one lineage-determining factor is OCT4, KLF4, SOX2, or MYC.

41. A method for performing cell transplantation in a recipient in need thereof, comprising
generating a second cell from a first cell of a donor, wherein said donor is immunocompatible with said recipient, and wherein said second cell is generated according to the method of any one of claims 1-26; and
transplanting said second cell into said recipient.

42. The method of claim 41, wherein said recipient and said donor are the same individual.

43. The method of claim 41 or 42, wherein said first cell is a fibroblast or an endothelial cell.

44. The method of any one of claims 41-43, wherein said second cell is a cardiomyocyte-like cell, a cardiomyocyte, a neuron, a skeletal muscle cell, or a stem cell-like cell.

45. A pharmaceutical composition comprising a first plurality of cells having a first cell type and media; made by a process comprising:
i) obtaining a second plurality of cells of a different cell type compared to the first cell type;
ii) reducing expression level(s) of at least one barrier gene selected from the group consisting of ATF7IP, JUNB, ZNF207, Sp7, FOXA1, HEXIM2, SMARCA5, SOX15, CHST2 and NCEH1 in the second plurality of cells; wherein when said at least one barrier gene is ATF7IP or SOX15, at least a second barrier gene is included; or
reducing expression level(s) of at least two barrier genes selected from the group consisting of ATF7IP, JUNB, ZNF207, and Sp7 in the second plurality of cells.

46. The pharmaceutical composition of claim 45, wherein said at least one barrier gene comprises ATF7IP, JUNB, and ZNF207.

47. The pharmaceutical composition of claim 46, wherein said at least one barrier gene further comprises Sp7.

48. The pharmaceutical composition of any one of claims 45-47, wherein said process further comprising increasing an expression level of at least one reprogramming agonist gene in the second plurality of cells.

49. The pharmaceutical composition of claim 48, wherein said at least one reprogramming agonist gene is selected from the group consisting of MEF2C, HSPB3, TPP1, EMC1, PPIC, IL7R, OLFML3, TCTA, and EFHD1.

50. The pharmaceutical composition of any one of claims 45-49, wherein said process further comprising increasing an expression level of at least one lineage-determining factor in the first plurality of cells.

51. The pharmaceutical composition of claim 50, wherein said expression level of said at least one lineage-determining factor is increased within 72 hours after said expression level(s) of said at least one barrier gene is reduced.

52. The pharmaceutical composition of claim 51, wherein said expression level of said at least one lineage-determining factor is increased within 48 hours after said expression level(s) of said at least one barrier gene is reduced.

53. The pharmaceutical composition of claim 52, wherein said expression level of said at least one lineage-determining factor is increased within 24 hours after said expression level(s) of said at least one barrier gene is reduced.

54. The pharmaceutical composition of any one of claims 45-53, wherein the second plurality of cells is a fibroblast, an endothelial cell, or a PBMC.

55. The pharmaceutical composition of any one of claims 45-54, wherein the process further comprising altering chromatin structure of the second plurality of cells.

56. The pharmaceutical composition of claim 55, wherein the second plurality of cells has a reduced number of motifs bound to a protein expressed by said at least one barrier gene compared to a wild-type cell of the second plurality of cells.

57. The pharmaceutical composition of claim 56, wherein said motifs comprise an AP-1 motif.

58. The pharmaceutical composition of any one of claims 45-57, wherein the first plurality of cells is a cardiomyocyte-like cell, a cardiomyocyte, a neuron, a skeletal muscle cell, or an induced pluripotent stem cell.

59. The pharmaceutical composition of any one of claims 50-58, wherein the at least one lineage-determining factor is selected from the group consisting of MEF2C, GATA4, TBX5, ASCL1, BRN2, MYTL1, OCT4, KLF4, SOX2, MYC, and MYOD.

60. The pharmaceutical composition of any one of claims 45-59, wherein the first plurality of cells has an increased expression of at least one cardiac marker as compared to the second plurality of cells.

61. The pharmaceutical composition of claim 60, wherein said at least one cardiac marker is selected from the group consisting of ACTC1, MYL7, TNNT2, SCN5A, RYR2, NPPA, and NPPB.

62. The pharmaceutical composition of any one of claims 45-61, wherein the first plurality of cells has an increased expression of at least one stem cell marker as compared to the second plurality of cells.

63. The pharmaceutical composition of claim 62, wherein said at least one stem cell marker is selected from the group consisting of SSEA4 and NANOG.

64. The pharmaceutical composition of any one of claims 45-63, wherein the first plurality of cells has an increased expression of at least one neuron-specific marker as compared to the second plurality of cells.

65. The pharmaceutical composition of claim 64, wherein said at least one neuron-specific marker is selected from the group consisting of vGLUT2, GAD67, PVALB, and SYN1.

66. The pharmaceutical composition of any one of claims 45-65, wherein said reducing expression level(s) of at least one barrier gene comprises contacting the second plurality of cells with at least one siRNA molecule targeting said at least one barrier gene.

67. The pharmaceutical composition of any one of claims 45-66, wherein the second plurality of cells are somatic cells.

68. The pharmaceutical composition of any one of claims 45-67, wherein the first plurality of cells are somatic cells.

69. The pharmaceutical composition of any one of claims 45-68, wherein the process further comprising converting the second plurality of cells into an induced pluripotent stem cell (iPSC) state.
